(19) United States
(12) Patent Application Publication
(10) Pub. No.: US 2010/0312008 A1
(43) Pub. Date: Dec. 9, 2010

(54) SOLID ACID CATALYSTS, METHODS OF MAKING, AND METHODS OF USE

(76) Inventors: James R. Kastner, Athens, GA (US); Dan Geller, Athens, GA (US); Lawrence H. Keith, Monroe, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/797,086

(22) Filed: Jun. 9, 2010

Related U.S. Application Data
(60) Provisional application No. 61/185,233, filed on Jun. 9, 2009.

(51) Int. Cl. C07C 69/02; C01B 25/02; C01B 31/08

(52) U.S. Cl. 560/231; 423/304; 423/415.1

(57) ABSTRACT

Embodiments of the present disclosure, in one aspect, relate to solid acid catalysts, methods of making solid acid catalysts, methods of using solid acid catalysts, and the like. An embodiment of the present disclosure can include a reusable and recoverable solid, carbon supported, porous acid catalyst for biodiesel generation using activated carbon or biochar generated from agricultural or forestry residues.

### CROSS-REFERENCE TO RELATED APPLICATION

This application claims priority to co-pending U.S. provisional application entitled “Solid Acid Catalysts and Methods of Making” having Ser. No. 61/185,233 filed on Jun. 9, 2009, which is entirely incorporated herein by reference.

### STATEMENT REGARDING FEDERALLY SPONSORED RESEARCH OR DEVELOPMENT

This invention was made with government support under the Small Business Innovation Research Program (SBIR Phase I) awarded by the U.S. EPA. The U.S. government has certain rights in the invention.

### BACKGROUND

The economics of biodiesel production is strongly linked to the cost of feedstock. In the U.S., the raw material cost of the feedstock (e.g., soybean oil) for biodiesel production is significantly more than the cost of petroleum based diesel. Feedstock cost accounts for the largest fraction of the cost for biodiesel production. Thus, the high cost of refined vegetable oil and the inability to recover/reuse the catalyst is a barrier to economically feasible biodiesel commercialization. Alternatively, inexpensive sources of triglycerides, such as yellow (<15% free fatty acid or FFA) or brown (>15% FFA) grease, rendered fat, and soapstock, can be used to generate biodiesel—all feedstocks high in free fatty acids or FFA's. Unlike acid catalysts, base catalysts (e.g., KOH) can't be used to generate biodiesel from inexpensive feedstocks due to the formation of soaps from the FFA's—thus, an acid catalyst is required. Recently, a two step process utilizing an acid catalyst (H₂SO₄) and methanol to esterify the FFA's followed by a base catalyst (KOH) to transesterify the triglycerides was demonstrated to effectively produce biodiesel from waste oils. A significant drawback to the two-step process is the need to recover or neutralize the homogeneous acid catalyst in stage one before the second stage. The development of heterogeneous (i.e., reactants and catalyst are in a separate phase; e.g., FFA and methanol in the liquid phase and a porous solid acid catalyst) acid and base catalysts capable of esterifying or transesterifying FFA's and triglycerides would solve critical problems in the commercial scale development of biodiesel from inexpensive lipid sources. Thus, there is significant need for the development of heterogeneous acid and base catalysts to perform both esterification and transesterication reactions for biodiesel production.

Recently, this awareness has resulted in research on the synthesis and testing of heterogeneous acid and base catalysts for esterification. Alkaline earth oxides, such as CaO and MgO, have been used to transesterify triglycerides with methanol from room temperature to 180° C. Nanoscale CaO crystals have been shown to significantly improve the process by reducing the time for maximum yield and lowering the operating temperature. However, deactivation of the catalyst occurred after repeated use due to impurity deposition, and there has been no research on the generation and use of supported base catalysts (e.g., dispersion of CaO nanoparticles in/on a stable, strong porous matrix that could be used in a large reactor) which would be required for scale-up.

Similarly, there have been recent reports on the generation of solid acid catalysts. Anion exchange resins (e.g., polystyrenesulphonic acid) have been used to esterify FFA's with a range of alcohols, yet are expensive and potentially unstable at high pH. Perfluorinated alkanes supported on silicon oxides catalyze esterification, but again are expensive, environmentally unfriendly, unstable at high pH, and are generated from non-renewable carbon sources. Heteropolyacids impregnated/attached on/to zirconia have also been developed, but this support material (i.e., zirconia) is very expensive.

### SUMMARY

Embodiments of the present disclosure relate to solid acid catalysts, methods of making solid acid catalysts, methods of using solid acid catalysts, and the like.

Briefly described, embodiments of the present disclosure can include a solid acid catalyst including a support and a plurality of acidic functional groups attached to the support.

Briefly described, embodiments of the present disclosure can include a method of making a solid acid catalyst including pyrolyzing biomass to form biochar and activating the biochar.

Embodiments of the present disclosure can include a method of using a solid acid catalyst including esterifying free fatty acids that are in contact with the solid acid catalyst for about 1 to 6 hours at about 25° C. to 60° C.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIGS. 1A-1B illustrate digital images of a pyrolysis unit (FIG. 1A) and resultant peanut hull char (FIG. 1B). FIG. 1C illustrates a microscopic scale of the char, the bar indicates 2 μm.

FIGS. 2A-2B are graphs that illustrate the effect of ozone treatment on NH₃ adsorption measured in breakthrough curves at 6 ppmv NH₃ for commercial activated carbon or AC, FIG. 2A: , untreated AC, 30 min ozone, ▴, replicate, and peanut hull char or PHC, FIG. 2B: , untreated PHC, ∘, 30 min ozone. Note, the shift to the right (arrow) indicates an increase in NH₃ adsorption. 6.1 L/min, 1-in diameter reactor, 5 g of char (1 inch height of char).

FIG. 3 is a graph that illustrates a comparison of catalytic ozonation activity for NH₃ oxidation between steam, pyrolyzed peanut hull char (S-PHC), ; pyrolyzed PHC, ∘; wood fly ash, ▪; and poultry litter ash, ▴; all at 23° C., 5 g catalyst, 6.35 L/min, 2.5 cm catalyst packing height, and a 10.7-10.9 ppmv NH₃ inlet.

FIG. 4 illustrates a batch pyrolysis unit used to generate chars for synthesis of solid acid catalysts.

FIG. 5 illustrates a schematic of the apparatus used to ozonate (generated acidic/oxygenated functional groups) on the char: 1) compressed O₂ source, 2) pressure regulator, 3) ozone generator, 4) packed-bed column with char, 5) ozone detector (not used in this work), and 6) outlet to hood.

FIG. 6 is a graph that illustrates DRIFT analysis of sulfonated peanut hull chars, generated 400 (blue), 500 (yellow), and 600° C. (pink) and sulfonated at 250° C. for 12 h.

FIG. 7 is a graph that illustrates a comparison of DRIFT analysis for sulfonated peanut hull (yellow) and pine pellet (blue) chars, generated 500° C. and sulfonated at 250° C. for 12 h.

FIG. 8 is a graph that illustrates the effect of pyrolysis temperature on sulfonation determined by DRIFT analysis for sulfonated peanut hull chars, generated at 500° C. and 400° C. both sulfonated at 150° C. for 12 h.

FIG. 9 is a graph that illustrates a comparison of DRIFT analysis for sulfonated pine chip char before (blue) and after repeated esterification reactions without regeneration (pink). Char was pyrolyzed at 400° C. and sulfonated at 100° C. for 12 h.

FIG. 10 is a graph that illustrates formation of fatty acid methyl esters from palmitic and stearic acids (200 ppm) using granular ozonated and sulfonated pine pellet char (55° C., 240 min reaction time, 500° C. char).

FIG. 11 is a graph that illustrates change in percent conversion of FFA's and formation of methylesters using granular and powdered ozonated and sulfonated pine pellet char (500° C. char).

FIG. 12 is a graph that illustrates change in percent conversion of FFA's and formation of methylesters using granular and powdered ozonated and sulfonated peanut hull char (500° C. char).

FIGS. 13A-13B are graphs that illustrate the effect of pyrolysis and sulfonation temperature on esterification activity using peanut hull char generated at 500° C. (♦) or 400° C. (▪) and sulfonated at 250° C. (♦) or 100° C. (▪). All reactions were performed at 60° C. with 0.5 g of sulfonated char for 30 minutes.

FIG. 14 is a graph that illustrates the effect of starting biomass structure (pyrolysis 400° C., sulfonation 100° C.) on esterification activity (palmitic and stearic) using either peanut hull (▴, x) or pine chip (▪, ♦) char. All reactions were performed at 60° C. with 0.5 g of sulfonated char for 30 minutes.

FIG. 15 is a graph that illustrates the effect of regeneration methods on esterification activity (stearic acid ▪, palmitic acid ♦) of solid acid char catalyst (pine chip char pyrolysis 400° C., sulfonation 100° C.). Regeneration methods included heating with heptane (A, heptane treatment) and just heating the catalyst at 125° C. for 1 h (B, drying only). All reactions were performed at 60° C. with 0.5 g of sulfonated char for 30 minutes. Note—Regeneration treatment was not performed on reuse of the char catalyst from treatments 1 to 7.

FIG. 16 is a graph that illustrates the effect of heating and drying the char catalyst as a regeneration method for esterification activity (stearic acid ▪, palmitic acid ♦; pine chip char pyrolysis 400° C., sulfonation 100° C.). In between treatments the char catalysts was heated at 125° C. for 1 h. All reactions were performed at 60° C. with 0.5 g of sulfonated char for 30 minutes.

FIG. 17 illustrates a schematic for batch pyrolysis unit (3) used to generate biochars for synthesis of solid acid carbon catalysts.

FIG. 18 is a graph that illustrates DRIFT analysis of peanut hull chars (PHC) generated at 400, 500, and 600° C. via pyrolysis and sulfonated at 250° C. for 12 h, as well as PHC generated at 400° C. (untreated). The DRIFT analysis was conducted in the Kubelka-Munk mode and is presented over the same range of intensity.

FIG. 19 is a graph that illustrates DRIFT analysis of peanut hull chars (PHC) generated at 400° C. via pyrolysis and sulfonated at 100, 150, and 250° C. for 12 h. The DRIFT analysis was conducted in the Kubelka-Munk mode and the signal ranged from 0-3 for all plots.

FIGS. 20A and 20B are graphs that illustrate DRIFT (FIG. 20A) and ATR (FIG. 20B) analysis of pine pellet char (PPC) and peanut hull (PHC) char generated at 400 or 500° C. via pyrolysis and sulfonated at 100 or 250° C. for 12 h compared to pine chips (PCC) pyrolyzed at 400° C. and sulfonated at 100° C. or a non-sulfonated control. The DRIFT analysis was conducted in the Kubelka-Munk mode and the signal ranged from 0-2.5 for pine pellet char and 0-6 for pine chip char (PCC).

FIGS. 21A-21D illustrate a comparison of esterification catalytic activity between sulfonated pelletized peanut hull and pine biochar (500° C. pyrolysis, sulfonation 250° C.). Reaction conditions were 200 ppm palmitic or stearic acid in methanol (5 ml), 0.20 g char, and 58° C.

FIGS. 22A-22B illustrate a comparison of esterification catalytic activity between granular and powdered sulfonated peanut hull (PHC) and pine pellet biochar (500° C. pyrolysis, sulfonation 250° C.). Reaction conditions were 200 ppm palmitic or stearic acid in methanol (5 ml), 0.20 g char, and 55° C.

FIGS. 23A-23B illustrate the effect of pyrolysis and sulfonation temperature on esterification catalytic activity (peanut hull char, 500° C. pyrolysis and 250° C. sulfonation; 400° C. pyrolysis and 100° C. sulfonation). Reaction conditions were 500 ppm palmitic and stearic acid, 0.50 g char, and 58° C. for 1 hour. Catalysts were recovered, rinsed with methanol and reused without further treatment

FIGS. 24A-24B illustrate a comparison of esterification catalytic activity between different sulfonated biochars. Reaction conditions were 500 ppm palmitic and stearic acid, 0.50 g char, and 58° C. for 1 hour. Catalysts were recovered, rinsed with methanol and reused without further treatment.

FIG. 25 is a graph that illustrates DRIFT analysis of acid catalysts after repeated reuse (7× with methanol rinsing only) for the catalytic esterification of palmitic. Pine chip char (PCC) was generated at 400° C. via pyrolysis and sulfonated at 100° C. for 12 h. The DRIFT analysis was conducted in the Kubelka-Munk mode and the signal ranged from 0-6 for both plots.

FIGS. 26A-26B illustrate the effect of heating the acid catalyst in heptane (top, 250° C.) or heating only (bottom, 125° C.) on esterification activity during reuse. The period before A indicates methanol rinsing only, after A indicates heptane rinsing following by heating at 250° C., and after B represents heptane rinsing followed by heating 125° C. (top). The catalyst was generated from pine chip biochar pyrolyzed at 400° C. and sulfonated at 100° C. for 12 h. All reactions were performed at 60° C. with 0.5 g of sulfonated char for 30 minutes. Note—Regeneration treatment was not performed on reuse of the char catalyst from treatments 1 to 6 (top).

FIGS. 27A-27B illustrate DRIFT analysis of pine chip char (PCC, A) and peanut hull char (PHC, B) chars generated at 400° C. via pyrolysis, pre-oxidized with ozone and functionalized with EDA at 100° C. for 12 h compared to their non-treated chars. The DRIFT analysis was conducted in the Kubelka-Munk mode, the EDA functional chars are presented as difference spectrums, the signal ranging from 0-2 for functional chars, and 0-0.25 for the ozonated chars, and 0-6 for the non-treated chars (i.e., biochar).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

“Biomass” can include products, by-products, and/or residues of the forestry and agriculture industries. Biomass includes, but is not limited to, plants, trees, crops, crop residues, grasses, forest and mill residues, wood and wood wastes, fast-growing trees, and combinations thereof. In particular, biomass can include pine trees (e.g., chips, shavings, and the like) and pine tree by-products and other tree or plant material. In an embodiment of the present disclosure, biomass can be selected from the group consisting of: a peanut hull, a wood chip, a pine pellet, and a combination thereof.

“Pyrolysis” is the thermal conversion of a base material, such as plant material, in the absence of oxygen at temperatures generally about 300 to 1300° C., about 300 to 1000° C., about 600 to 900° C., or about 800° C. When treated at these temperatures, the base material is carbonized to form biochar.

“Biochar” is a carbonized form of a plant material (e.g., bedding material) that is specifically produced for non-fuel applications. The process of production gives biochar properties that make it suitable for applications such as adsorption, enhancing microbial activity, and the like. Production processes can be batch or continuous, where base material of particle sizes ranging from about a few millimeters to several centimeters are placed in a retort, with or without carrier gas flowing through. Carrier gases may be non-reactive such as nitrogen, or reactive such as steam. The retort may be heated by external heat or directly heated by combusting a portion of the base material. Vapors emanating may be captured for other applications. After a period of several minutes to hours, the residual material remaining is biochar. Biochar is composed of mainly carbon (e.g., about 30 to 100% or about 60 to 100%) and is porous. Other elements (such as nitrogen, oxygen, hydrogen) are gradually lost at elevated temperatures. The molecular structure and elemental composition makes biochar highly recalcitrant against microbial decomposition.

### Discussion:

In accordance with the purpose(s) of the present disclosure, as embodied and broadly described herein, embodiments of the present disclosure, in one aspect, relate to solid acid catalysts, methods of making solid acid catalysts, methods of using solid acid catalysts, and the like. An embodiment of the present disclosure can include a reusable and recoverable solid, carbon supported, porous (e.g., large surface area available for chemical reactions) acid catalyst for biodiesel generation using activated carbon or biochar generated from agricultural or forestry residues. In another embodiment of the present disclosure, the catalyst can be capable of catalyzing esterification reactions using waste lipids (e.g., FFA's>15%) without deactivation and at temperatures about 150° C. or lower.

Carbon supported catalysts have several distinct advantages over alumina or silica supported systems: 1) they are stable under acidic and basic conditions, as well as high temperature (e.g., about 200-300° C.) and can have very high surface area (e.g., about 200-1500 m²/g), 2) active material (e.g., acidic functional groups) can be finely dispersed (e.g., large surface area of the carbon support is available for chemical reactions) throughout the carbon structure increasing accessibility of reactants to the catalyst relative to a bulk catalyst, 3) the amount of active material needed for catalyst development is reduced, 4) a renewable carbon source can be used to generate the active carbon, or 5) the non-polar nature of the support matrix may prevent adsorption of polar molecules (e.g., water or glycerol) that can deactivate the catalyst in the transesterification/esterification of lipids

An embodiment of the present disclosure can include a method of making a solid acid catalyst where acidic functional groups are attached (e.g., covalently and/or ionically) to biochar, carbon, or activated carbon, subsequently transforming the carbon into a solid acid catalyst. The solid acid, carbon supported catalysts can be used in a wide variety of industrial processes, including, but not limited to, the production of biodiesel from high free fatty acid oils and fats or industrial feedstocks of free fatty acids only. In addition, the solid acid catalysts may be used for stabilization of bio-oils for liquid fuels, conversion of methanol to dimethylether (DME) a biodiesel substitute, cellulose hydrolysis to glucose and subsequent fermentation to liquid fuels (e.g., ethanol, isobutanol).

An embodiment of the present disclosure can include a solid acid catalyst including a support and a plurality of acidic functional groups attached to the support. In an embodiment, the support is selected from the group consisting of: biochar, carbon, activated carbon, and a combination thereof. In another embodiment, the support is derived from biomass. In another embodiment, the biomass is selected from the group consisting of: peanut hull, wood chip, pine pellet, and a combination thereof.

Activated carbon or char from biomass, as previously noted, has multiple advantages as a catalyst support over other materials (e.g., potential for high surface area resulting in higher rates, pH and temperature stability, minimizes catalyst deactivation), is a renewable material, is more economical than refined sugar (e.g., granulated sugar or starch) or synthetic polymer derived from petroleum as a feedstock for a carbon support, and will result in a co-product from biorefineries.

An embodiment of the present disclosure can include a solid acid catalyst including acidic functional groups selected from the group consisting of: —SO₃H, —PO₃, and a combination thereof.

Embodiments of the present disclosure can include a reusable solid acid catalyst. Reusable catalysts are used in batch mode for one reaction cycle, recovered, washed, heated, and then used in another reaction cycle (e.g., used about 1 to 7 times).

Embodiments of the present disclosure can include a method of making a solid acid catalyst including pyrolyzing biomass to form biochar and subsequently activating the biochar. In an embodiment, the biomass is pyrolyzed at about 400° C. to 600° C. In another embodiment, the biochar is activating with H₂SO₄ (e.g., 99% H₂SO₄, fuming sulfuric acid, SO₃ gas). In another embodiment, the biochar is activated at about 50° C. to 250° C. In another embodiment, the biochar is activated at about 100° C. to 250° C.

An embodiment of the present disclosure can include a method of using a solid acid catalyst including drying the solid acid catalyst at about 100 to 125° C. for about 1 hour between uses to allow the solid acid catalyst to be reused.

An embodiment of the present disclosure can include a method of using a solid acid catalyst including esterifying free fatty acids that are in contact with the solid acid catalyst for about 1 to 6 hours at about 25° C. to 60° C.

Embodiments of the present disclosure can be used in hydrolysis of biomass or lignocellulosic cellulose and hemicelluloses to sugars.

Embodiments of the present disclosure can also be used in the catalytic esterification of alcohols (e.g., methanol, ethanol, and glycerol) with low molecular weight carboxylic acids in bio-oil (acetic and formic) to form methylesters and stabilize bio-oil for transportation. Bio-oil is oil made by pyrolysis of biomass.

In addition, embodiments of the present disclosure can be used for catalytic esterification of acetic acid and glycerol to form triacetin using solid acid catalyst made from biochar.

### EXAMPLES

### Example 1

A reusable solid acid carbon supported catalyst was generated from peanut hull, wood chip, and pine pellet biochar. Catalysts were generated by pyrolyzing peanut hull pellets, wood chips, and pine pellets at 400 and 500° C. to generate a soft to hard carbon backbone for addition of acidic functional groups. Next, the carbonized materials were sulfonated using concentrated H₂SO₄ at 100, 150 and 250° C. (12 h) and subsequently washed and dried to generate a solid acid catalyst (—SO₃H attached to carbon backbone). DRIFT analysis of the sulfonated chars indicated the presence of —SO₃ groups on the 100° C. sulfonated wood chip char (400° C. pyrolysis). The sulfonated biochars were subsequently tested for their ability to esterify free fatty acids (palmitic acid and stearic acids) with methanol. Esterification of the fatty acids was typically complete (˜90-100% conversion) within 1 hour at 60° C. or 6 hours at 25° C. Of the synthesized catalysts, 400° C. pyrolyzed pine chip char, sulfonated at 100° C., resulted in the highest reaction rate and lowest reduction in conversion (or deactivation) when reused multiple times. Simply drying the char based acid catalysts for 1 hour at 125° C. between uses maintained esterification activity, thus allowing the catalysts to be reused (tested up to 7×).

### Outcomes:

A solid acid catalyst generated from biomass char would overcome some of the major problems in biodiesel production, including inefficiencies associated with a semi-continuous or continuous process, inability to use low cost/low quality oils high in free fatty acids as feedstock (e.g., yellow grease or rendered fat), the use of homogeneous catalysts (e.g., KOH/H₂SO₄) which must be continuously added and are not recovered, the production of residual waste streams that must be pH adjusted, and the inability to perform esterification in the presence of moisture. A further advantage is that the catalyst would be developed from renewable carbon sources (e.g., pine, peanut hulls) adding to the potential suite of products produced in a biorefinery from agricultural and forestry products.

### Introduction

The economics of biodiesel production is strongly linked to the cost of the feedstock. In the US, the raw material cost of the feedstock (e.g., soybean oil) for biodiesel production is significantly more than the cost of petroleum based diesel. Feedstock cost accounts for the largest fraction of the cost for biodiesel production (Fuel Processing Technology. 2005. 86, 1087-1096; Ind. Eng. Chem. Res. 2006, 45:2901-2913, which are herein incorporated by reference for the corresponding discussion). Thus, the high cost of refined vegetable oil and the inability to recover/reuse the catalyst is a barrier to economically feasible biodiesel commercialization. Alternatively, inexpensive sources of triglycerides, such as yellow (<15% free fatty acid or FFA) or brown (>15% FFA) grease, rendered fat, and soapstock, can be used to generate biodiesel—all feedstocks high in free fatty acids or FFA's (Fuel Processing Technology. 2005. 86, 1087-1096; Kulkarni, M G; Ind. Eng. Chem. Res. 2006, 45:2901-2913, which are herein incorporated by reference for the corresponding discussion). Unlike acid catalysts, base catalysts (e.g., KOH) can't be used to generate biodiesel from inexpensive feedstocks due to the formation of soaps from the FFA's—thus, an acid catalyst is required (Ind. Eng. Chem. Res. 2006, 45:2901-2913, which is herein incorporated by reference for the corresponding discussion).

Recently, a two step process utilizing an acid catalyst (H₂SO₄) and methanol to esterify the FFA's followed by a base catalyst (KOH) to transesterify the triglycerides was demonstrated to effectively produce biodiesel from waste oils (Id.). A significant drawback to the two-step process is the need to recover or neutralize the homogeneous acid catalyst in stage one before the second stage. The development of heterogeneous (i.e., reactants and catalyst are in a separate phase; e.g., FFA and methanol in the liquid phase and a porous solid acid catalyst) acid and base catalysts capable of esterifying or transesterifying FFA's and triglycerides would solve critical problems in the commercial scale development of biodiesel from inexpensive lipid sources (Ind. Eng. Chem. Res. 2005, 44:5353-5363, which is herein incorporated by reference for the corresponding discussion). For example, one could now envision a continuous, two-step process (e.g., 2 CSTR's in series) with a solid acid catalyst immobilized/retained in one reactor followed by a second reactor with a solid base catalyst. Thus, there is significant need for the development of heterogeneous acid and base catalysts to perform both esterification and transesterication reactions for biodiesel production.

This awareness has resulted in research on the synthesis and testing of heterogeneous acid and base catalysts for esterification. Alkaline earth oxides, such as CaO and MgO, have been used to transesterify triglycerides with methanol from room temperature to 180° C. (Energy and Fuels 2006, 20, 1310-1314; Industrial and Engineering Chemistry Research 2006, 45, 3009-3014). Nanoscale CaO crystals were shown to significantly improve the process by reducing the time for maximum yield and lowering the operating temperature (Energy and Fuels 2006, 20, 1310-1314). However, deactivation of the catalyst occurred after repeated use due to impurity deposition and there has been no research on the generation and use of supported base catalysts (e.g., dispersion of CaO nanoparticles in/on a stable, strong porous matrix that could be used in a large reactor) which would be required for scale-up.

Similarly, there have been recent reports on the generation of solid acid catalysts. Anion exchange resins (e.g., polystyrenesulphonic acid) have been used to esterify FFA's with a range of alcohols, yet are expensive and potentially unstable at high pH (Bioresource Technology 2007, 98, 2, 416-421). Perfluorinated alkanes supported on silicon oxides catalyze esterification (Journal of Catalysis 2007, 245, 2, 381-391), but again are expensive, environmentally unfriendly, unstable at high pH, and are generated from non-renewable carbon sources. Heteropolyacids impregnated/attached on/to zirconia have also been developed, but this support material (i.e., zirconia) is very expensive (Green Chemistry. 2006, 45:2901-2913, which is herein incorporated by reference for the corresponding discussion).

An option that has great possibility, but has not been fully explored, is the generation of acid or base catalysts supported on activated carbon or char for catalytic esterification/transesterification. Carbon supported catalysts have several distinct advantages over alumina or silica supported systems; 1) they are stable under acidic and basic conditions, as well as high temperature (200-300° C.) and can have very high surface area [200-1500 m²/g], 2) active material (e.g., acidic functional groups) can be finely dispersed throughout the carbon structure increasing accessibility of reactants to the catalyst relative to a bulk catalyst, 3) the amount of active material needed for catalyst development is reduced, 4) a renewable carbon source can be used to generate the active carbon, and 5) the non-polar nature of the support matrix may prevent adsorption of polar molecules (e.g., water or glycerol) that can deactivate the catalyst in transesterification/esterification of lipids (Applied Catalysis A: General. 1998, 173:259-271; Applied Catalysis A: General. 1998, 173:273-287; Ind. Eng. Chem. Res. 2005, 44:5353-5363, which are herein incorporated by reference for the corresponding discussion). Recently, functionalized activated carbon (i.e., attached —SO₃H groups) has been generated from refined sugar and was demonstrated to catalyze the transesterification of oleic and stearic acid with ethanol (Nature. 2005, 438 (10): 178, which is herein incorporated by reference for the corresponding discussion). Both acidic and basic functional groups have been attached to wood based activated carbon, but these materials have not been tested for their ability to catalyze transesterification/esterification reactions (Separation Science and Technology. 2004, 39 (14): 3263-3279, which is herein incorporated by reference for the corresponding discussion).

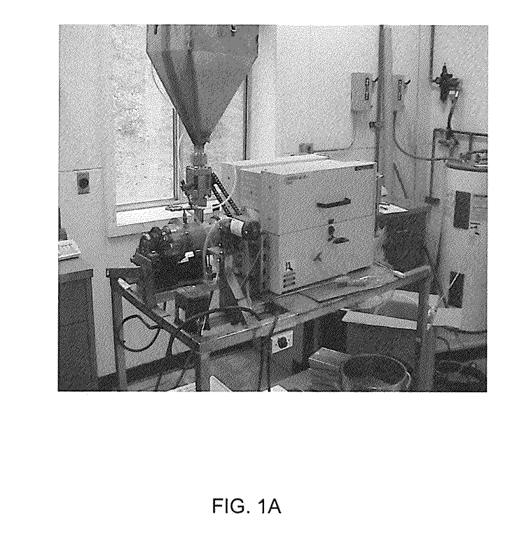

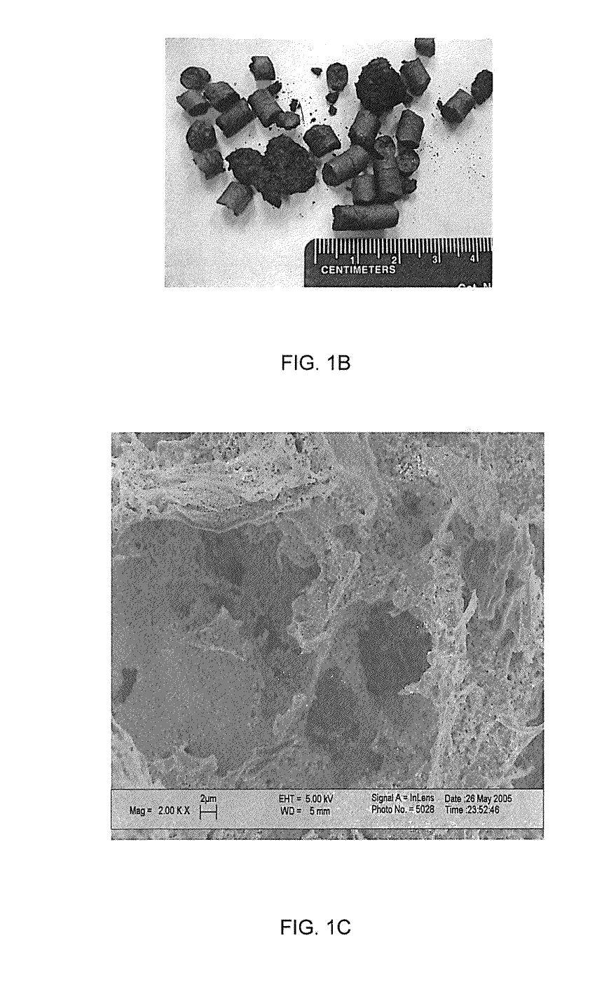

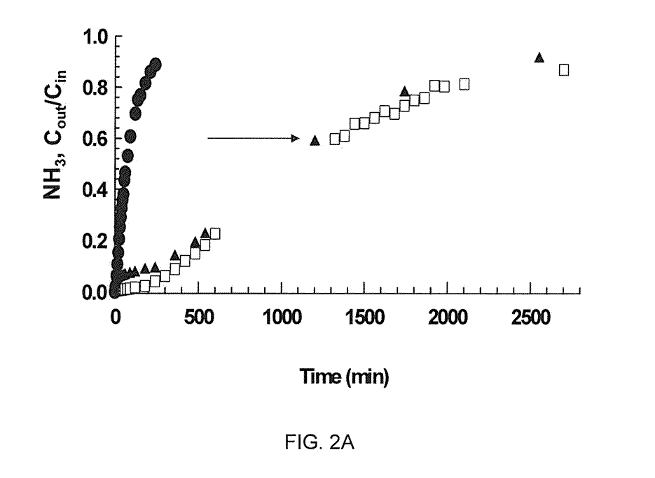

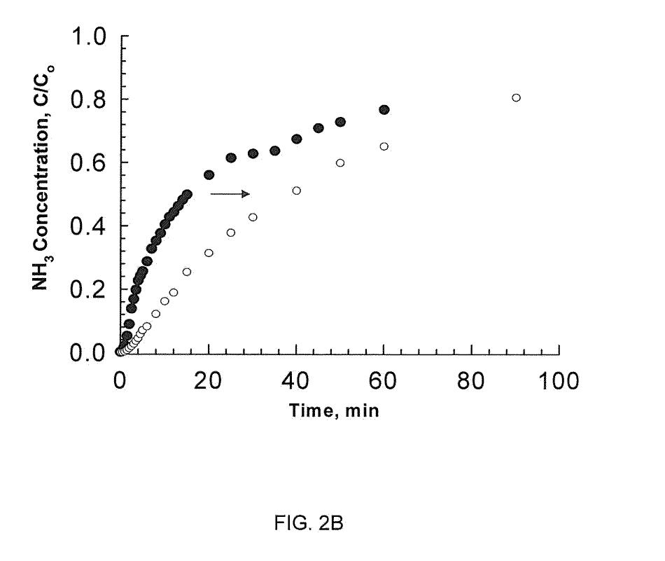

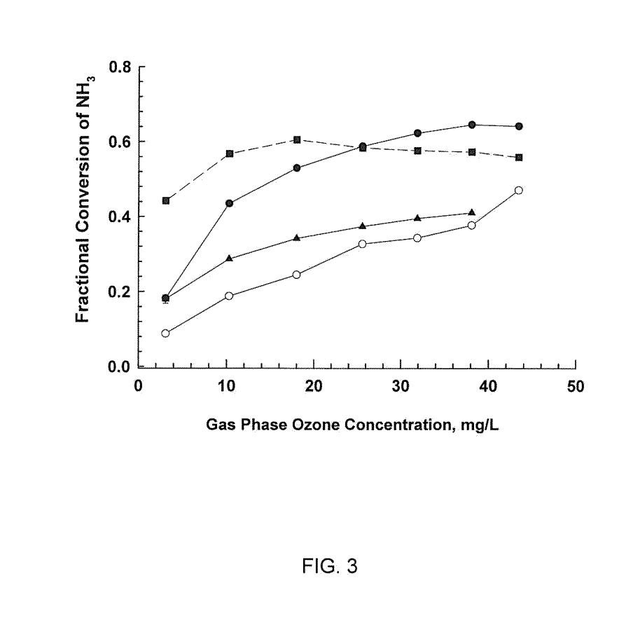

Our research group has recently been working on the development of functionalized activated carbon generated from agricultural residues that could be used to generate solid acid or base carbon supported catalysts. We have been generating char from peanut hulls (FIG. 1), subsequently activating the char using steam, and further functionalizing the carbon using ozone. The resultant carbons were shown to significantly increase ammonia adsorption (FIG. 2). Previous research in our group suggests that pyrolysis temperatures ranging from 400-700° C. should be tested and holding times longer than 30 minutes (1-3 hours) should increase the surface area of the char (increasing surface area typically increases adsorption and reaction rates).

Without being bound by any particular theory, we believe that the ozone treatment of the carbon generated acidic functional groups on the surface and in the pores of the char resulting in increased adsorption of NH₃ and demonstrates the ability of our group to generate functionalized carbon from biomass.

### Rationale and Significance:

Activated carbon or char from biomass, as previously noted, has multiple advantages as a catalyst support over other materials (e.g., potential for high surface area resulting in higher rates, pH and temperature stability, minimizes catalyst deactivation), is a renewable material, is more economical than refined sugar (e.g., granulated sugar or starch) or synthetic polymer derived from petroleum as a feedstock for a carbon support, and will result in a co-product from biorefineries. A heterogeneous acid catalyst for biodiesel synthesis from free fatty acids would reduce operating costs (catalyst recovery and cheap source of lipids) and increase productivity (allow for a continuous process). Finally, using solid acid and base catalysts in a two-stage process would allow for rapid esterification of FFA's in stage one (acid catalyst) followed by transesterification in stage two (base catalyst). The two-stage heterogeneous process would allow for optimization of methanol levels and reduce residence time (and thus reactor size), since the reaction conditions can be tailored toward the type of catalyst and substrate (i.e., FFA or triglyceride).

### Economic Justification:

The high cost of refined vegetable oil and the inability to recover/reuse the currently used homogeneous catalysts (either acid or base) is a barrier to biodiesel commercialization (Fuel Processing Technology. 2005. 86, 1087-1096; Ind. Eng. Chem. Res. 2006, 45:2901-2913, which are herein incorporated by reference for the corresponding discussion). This necessitates the need to use lipid sources with high concentrations of FFA's and thus an acid catalyst. Use of a heterogeneous acid catalyst could significantly reduce materials cost (acid resistant reactors and material would not be needed) and reduce the cost of neutralizing the acidic effluents (Bioresource Technology. 2003, 90: 229-240, which is herein incorporated by reference for the corresponding discussion). The use of non-polar, carbon based supported catalyst may also eliminate or reduce catalyst deactivation by water and glycerol and thus eliminate the need for removal of these two components in a two-stage, two-catalyst process (Bioresource Technology. 2003, 90: 229-240; Ind. Eng. Chem. Res. 2005, 44:5353-5363, which are herein incorporated by reference for the corresponding discussion).

### Experimental Approach:

Biomass Sources. Pelletized peanut hulls were supplied by Golden Peanut Co. LLC. Pelletized pine pellets and wood chips were purchased from local suppliers.

Char Generation. Pelletized peanut hulls (supplied by Golden Peanut Co., LLC, Alpharetta, Ga.) and pine logging residues, and wood chips, were used to generate char. The pelletized peanut hulls were typically 0.5-1 inches (1.3-2.54 cm) in length and 0.3125 inches (0.8 cm) in diameter. The composition of typical pelletized peanut hulls has been reported to consist of 2-4% ash, 35-45% cellulose, and 27-33% lignin, with a bulk density ranging from 609-720 kg m⁻³ and pH 5-6 (Johnson et al., and data provided by Golden Peanut Co., LLC). In addition to peanut hulls, pelletized pine and clean pine (i.e., wood) chips were used as feed stocks for pyrolysis and char generation and were analyzed in labs at UGA. More details on composition analysis of the biomass feedstock used to generate the char based catalyst are noted in Table 6.

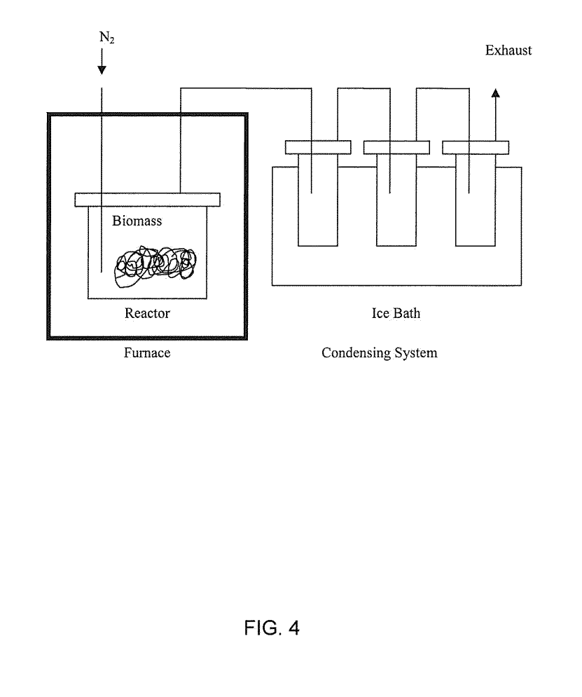

The pyrolysis unit consisted of a gas preheater (¼ OD inch stainless steel tube wrapped in heating tape (Thermolyne, Barnstead Inc, La) and was packed with fine steel mesh to provide turbulent mixing), followed by a tube furnace and condensation unit (FIG. 4). Preheat temperature was controlled via a solid-state relay connection to a maximum temperature of 700° C. The furnace was a tube type (1 in ID, ×16 in length, Type 55035, Lindberg, WS), with an actual heating length of 12 inches and installed thermocouple for temperature control. Pure nitrogen was used as an inert sweep gas and controlled using a mass flow controller. In this experiment, char was prepared over a range of temperatures, including 400, 500, and 600° C. and held for 40 min in the pyrolysis reactor with a N₂ flow at 200 ml min⁻¹.

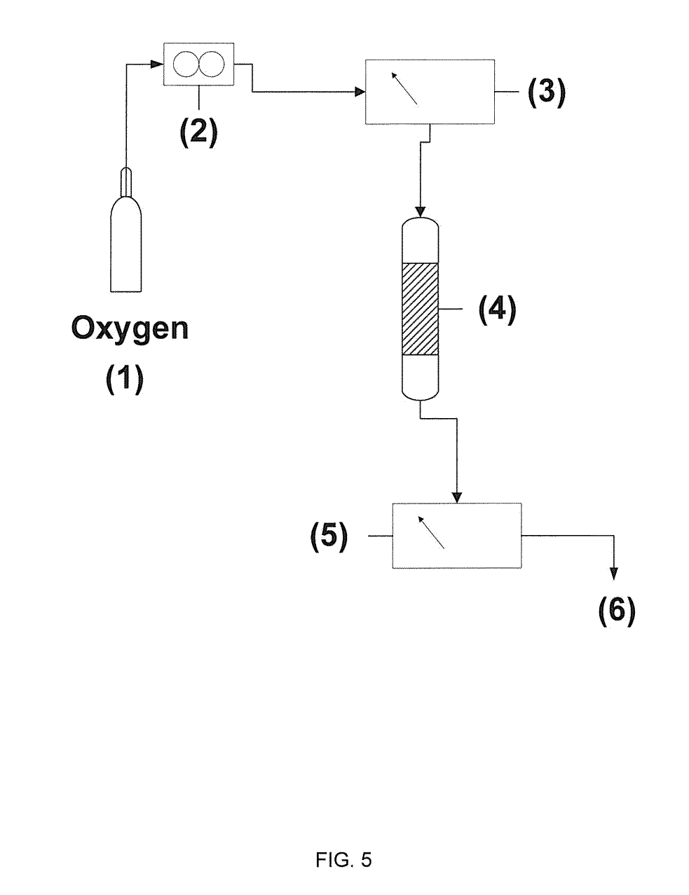

Ozone Treatment. In some cases, char was exposed to ozone in a fixed-bed reactor (1 in ID×12 mL, FIG. 5) packed with a defined mass of the char (25 g). Ozone in air was passed downward across the carbon at 6.1 L min⁻¹ (total flow) and 33 g m⁻³ O₃ for a period of 6 hours (23° C.). An ozone generator (OL100H/DS, Yanco Industries Ltd., B.C., Canada), utilizing a high frequency corona discharge, was used with a medium grade tank of oxygen (99.9%, National Wielders, N.C.) to generate the ozone required for these experiments.

Catalyst Generation: Next, the char generated above was further activated or reacted to attach or generate acidic functional groups on the carbon surface.

Acid Catalysts: Acid catalysts included, A-1, a weak acid functionalized carbon (char treated with ozone only) and, A-2, a strong acid —SO₃H functionalized char. For acid catalyst A-1, the char was oxidized using ozone at room temperature in a packed-bed reactor. We anticipate that this process introduced a high density of acidic groups (e.g., carboxylic acids, phenolic, and lactonic groups) throughout the carbon pores—this conclusion is based on our recent NH₃ adsorption studies (Journal of Hazardous Materials. 2009, 164, 1420-1427; also see FIG. 2).

For acid catalyst A-2 (—SO₃H), the chars (400, 500, and 600° C.) were sulfonated based on analysis of a range of methods described in the literature (Separation Science and Technology. 2004, 39 (14): 3263-3279; Nature. 2005, 438 (10): 178, which are herein incorporated by reference for the corresponding discussion). First, the pelletized chars were ground into smaller particles using a mortar and pestle (wood chip char was used as is). Pellets were ground and then sieved on 4-12 mesh screen sieves, retaining all char particles between in that specific range (Table 2). The sieved char was then weighed (25 g) and placed in an ACE glass reactor with glass wool and mesh screen “plug” to keep the char in place. Ozonation of the char was accomplished with a Yanco Ozone Generator at a concentration of 33 g/m³ for a total of 6 hours.

Subsequently, 12.5 grams of the previously ozonated char or untreated char, was placed in a beaker, and contacted with 20 ml of concentrated sulfuric acid (99% H₂SO₄). The acid was mixed with the char via periodic stirring (15 min), and then excess acid was decanted. The residual wet solids (char plus acid) were then transferred to a ceramic crucible, placed in muffle furnace, and heated for 12-18 hours at 100, 150, and 250° C. After heating, the char was cooled, rinsed 2-10× with 50-100 ml of deionized water. The pH of the rinsate was measured after each washing and the char was then dried in an oven at 110° C. overnight.

Catalyst Characterization: The physical and chemical characteristics of the chars, including pH, surface area, bulk density, and the elemental composition were previously determined (Journal of Hazardous Materials. 2009, 164, 1420-1427) and are reported in Table 1.

To qualitatively assess the formation of functional groups on the surface of the treated char, FTIR analysis was performed on the base chars (i.e., just pyrolyzed) and the sulfonated chars. The chars were partially crushed and analyzed directly using a Diffuse Reflectance Infrared Fourier Transform or DRIFT cell (Varian 2000 FT-IR spectrometer, Scimitar Series). All generated catalysts were analyzed in triplicate. The DRIFT cell sample cup was completely packed with particles of the char and sulfonated char and leveled before analysis (the particles were not crushed to a fine powder and KBr was not used). The chars were not ground to fine powder and mixed with KBr because previous analysis indicated that this procedure can eliminate signals due to dilution of key functional groups on the surface of treated material, especially for granular activated carbon or similar materials (Chem. Mater. 1997, 9, 176-183, which is herein incorporated by reference for the corresponding discussion).

The DRIFT accessory is mounted on a base plate which employs kinematic pins to reproducibly position it in the sample compartment. The two slide mounts provide measurements for up to 4 samples, optimizing sampling efficiency. The optical path of the accessory uses four flat and two aspherical mirrors which are off-axis ellipsoids that cover a solid angle of 2π steradians over the sample, and form a beam condenser with a nominal factor of 5. The rugged design that comes pre-aligned, requiring no user adjustments, assuring convenient sampling, reproducible data collection, and reliable operation.

For each analysis, a base char (i.e., un-sulfonated) was used to generate a reference spectrum and subtracted from the FTIR spectrum of the sulfonated chars. The base char and sulfonated chars (˜20 mg) were scanned from 600 to 3500 wavenumbers (cm⁻¹) at a total of 64 scans and a resolution of 4 cm⁻¹. The DRIFT spectrum was output in the Kubelka-Munk form.

In the future, the generated catalysts will be physically characterized by measuring surface area (BET, N₂), X-ray diffraction analysis or XRD, SEM, and Energy Dispersive Analysis (EDS). XRD will tell us if crystalline structures have been formed and their structure, SEM will give information on the nano-structure of the catalyst and EDS will tell us what elements are present on the surface. These data will be helpful in correlating physical structure with catalytic activity and provide information on how to enhance the preparation process to increase activity.

Esterification Activity: Each catalyst was tested for its ability to form methyl esters using free fatty acids (palmitic and stearic acids) and methanol. The catalysts were tested in a small scale batch reactor system (Reacti-Therm, Pierce—Thermo Scientific). The Reacti-Therm was pre-calibrated to determine temperature output versus setting input. Pure palmitic and stearic acids were used as model free fatty acids and purchased for use (Sigma Aldrich or Fisher Scientific).

A known amount of catalyst (0.2 g) was charged into a vial (5 ml total volume) with a known initial volume of palmitic (C₁₆-saturated FFA) or stearic (C₁₈-saturated, both at 200 ppm) and methanol (4 ml). The mixture was then heated at 55-60° C. and sub-samples taken as function of time to determine the formation of methylesters. Control reactions consisted of the untreated char (negative control) or use of HCl (positive control). The liquid sub-samples were analyzed and quantified using a GC/FID and hexadecane as an internal standard to determine the concentration of methylesters formed. Fractional removal or % conversion of palmitic or stearic acid were based on the defined initial concentrations of the FFA's (maximum theoretical amount of methylester that could form) and the concentration of the methylesters of the FFA's that formed during the catalytic reaction,
% X or Conversion=[C_{final}(FFA-ME)]/Cᵢₙᵢₜᵢₐₗ(FFA-ME)×100, C is concentration, FFA is free fatty acid, and FFA-ME is fatty acid methylester.

### Results:

Catalyst Characterization: Chars formed at low temperature (<700° C.) and relatively short holding times (<1 h) typically have low surface area. As seen in Table 1, the generated chars had surface areas ranging from 1-6 m²/g, much lower than typical activated carbon (500-1,500 m²/g). However, it must be pointed out that high surface is not a prerequisite for successful catalysts, especially if the process of interest occurs under diffusion limited conditions. In those cases, much of the surface area is not utilized, and the cost and engineering effort to generate the higher surface is not economically feasible.

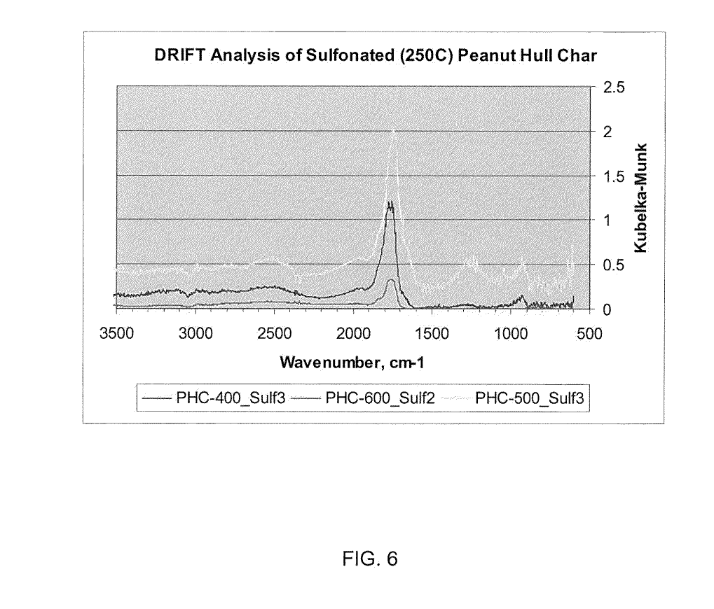

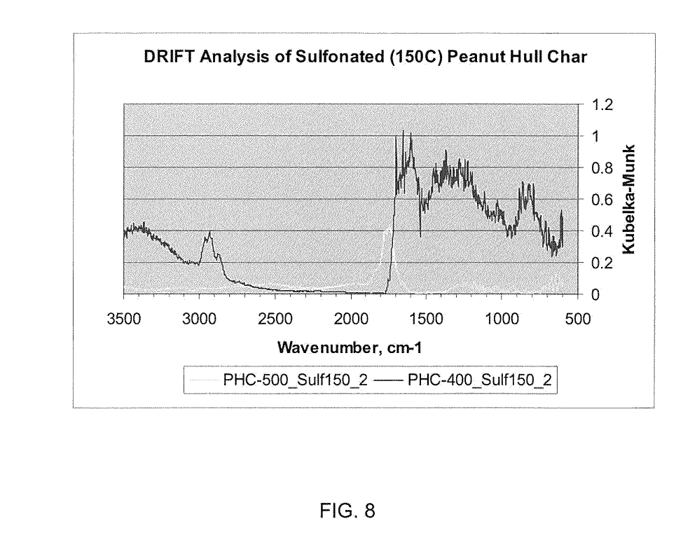

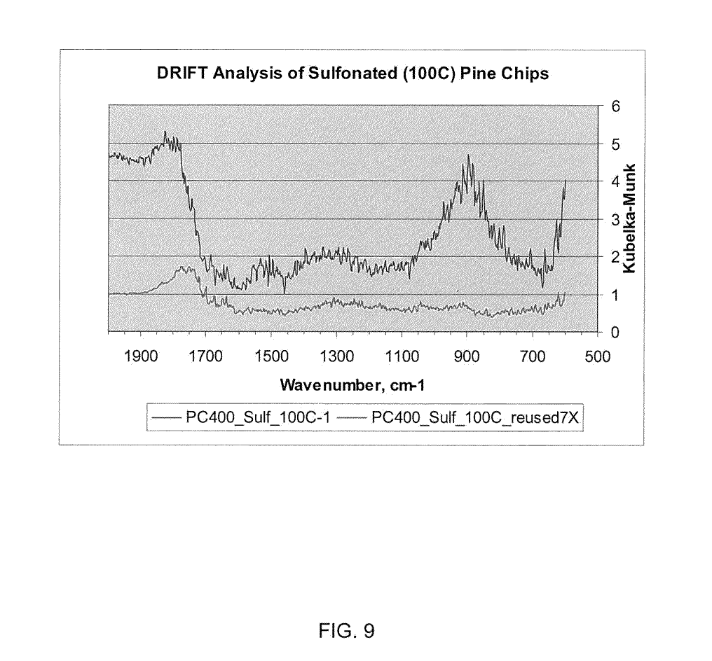

DRIFT Analysis: In general, based on DRIFT analysis, it appears that lower pyrolysis temperatures increased the reactivity of the chars towards sulfonation and lower sulfonation temperatures prevented over oxidation of the char (indicated by the appearance of a carboxylic acid peak at higher temperatures [e.g., 250° C.] and disappearance at lower temperature [100° C.]) and enhanced the formation of —SO₃ groups on the char surface. A large peak at 1750 cm⁻¹, indicative of a carboxylic acid group (Table 3) appeared in the sulfonated peanut hull chars generated at 400, 500, and 600° C. and a sulfonation temperature of 250° C. The peak intensity (1750 cm⁻¹) declined as the pyrolysis temperature of the formed chars increased (FIG. 6). Peaks suggestive of —SO₃ formation did not appear unless the pyrolysis and sulfonation temperatures were reduced (pyrolysis from 600 to 400° C. and sulfonation from 250 to 100° C.—for peanut hulls and pine)—this is demonstrated in FIG. 8 (and also FIG. 9 for pine, wood chip char) where the peak at 1750 cm⁻¹ disappears and broader less distinctive peaks at 1550-1600 cm⁻¹, 1100-1500 cm⁻¹, and 700-900 cm⁻¹ appear. It has been suggested that peaks at 1037, 1040, and 1200 cm⁻¹ are indicative of S=0 and —SO₃ groups (Table 3).

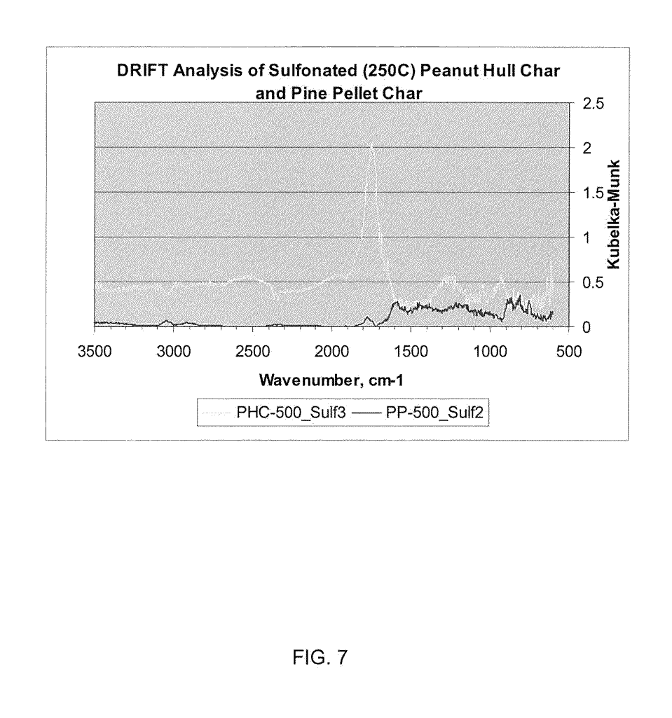

There was also a distinctive difference in the resultant DRIFT spectrum between pelletized peanut hull char and pine char. Contrary to peanut hull char (500° C.), pine pellet char (500° C.) sulfonated at 25° C. did not show evidence of a distinctive carboxylic acid peak, yet small peaks at ˜750, 820, 875 cm⁻¹ formed (FIG. 7). The formation of peaks most indicative of —SO₃ groups occurred when pine wood chips pyrolyzed at 400° C. were sulfonated at 100° C. (FIG. 9).

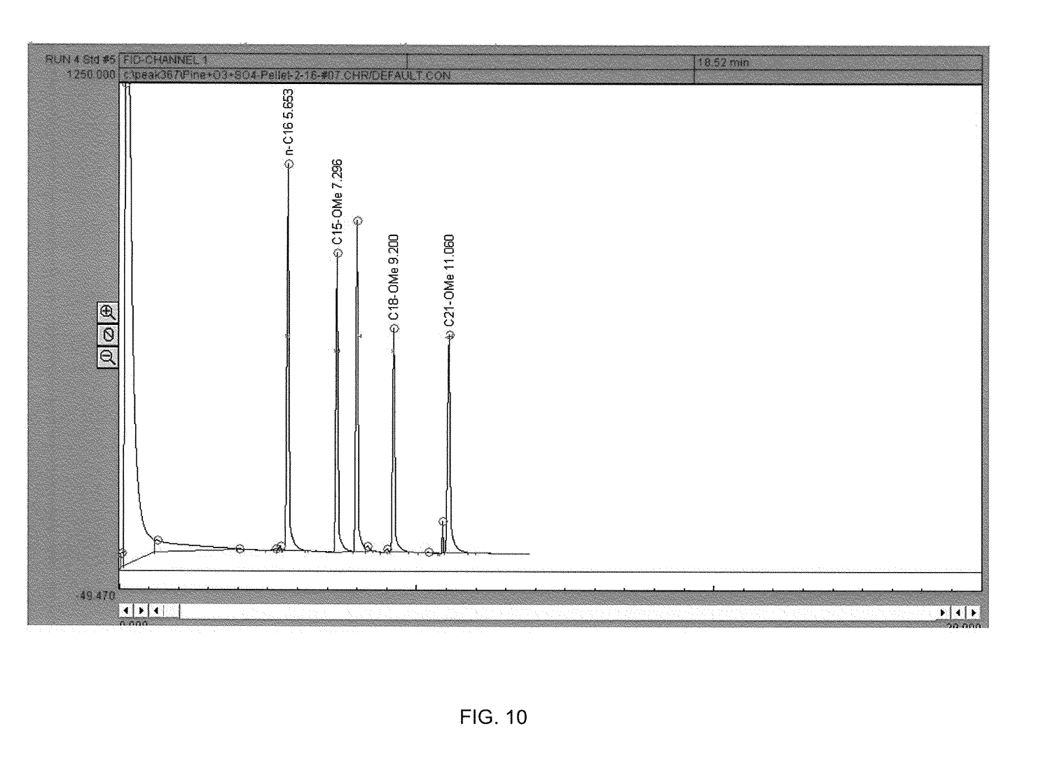

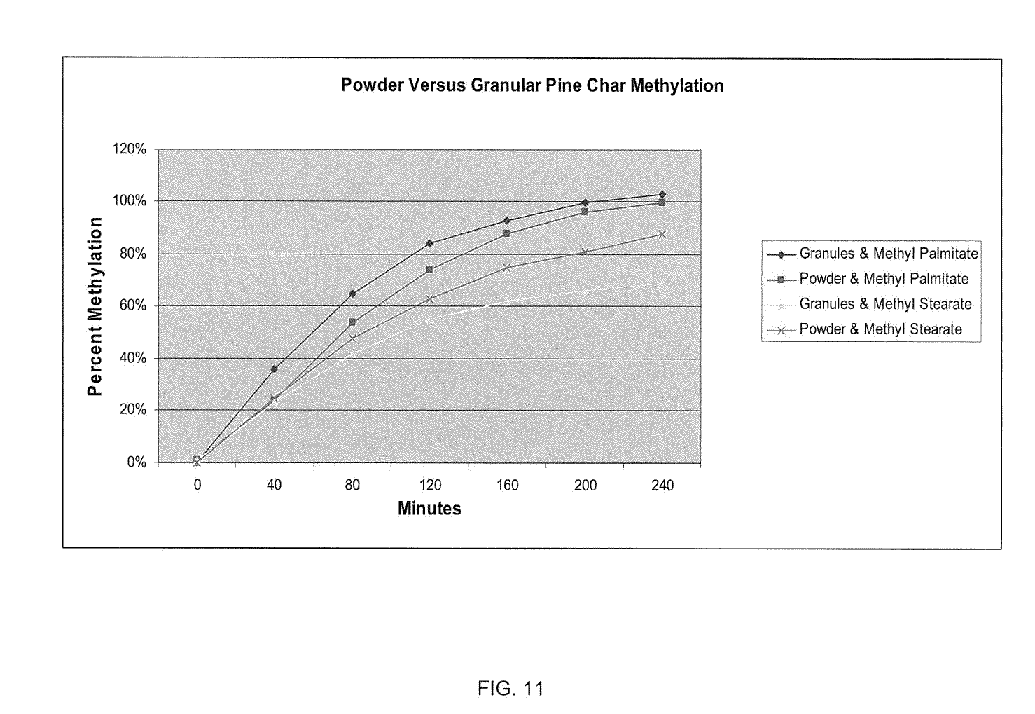

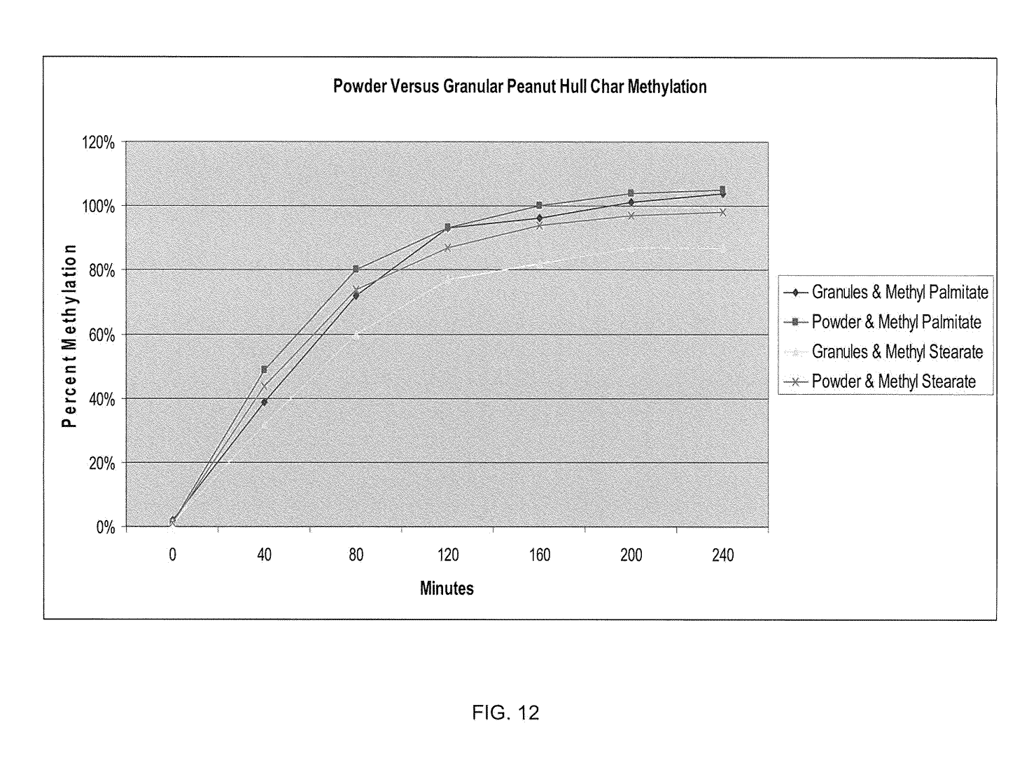

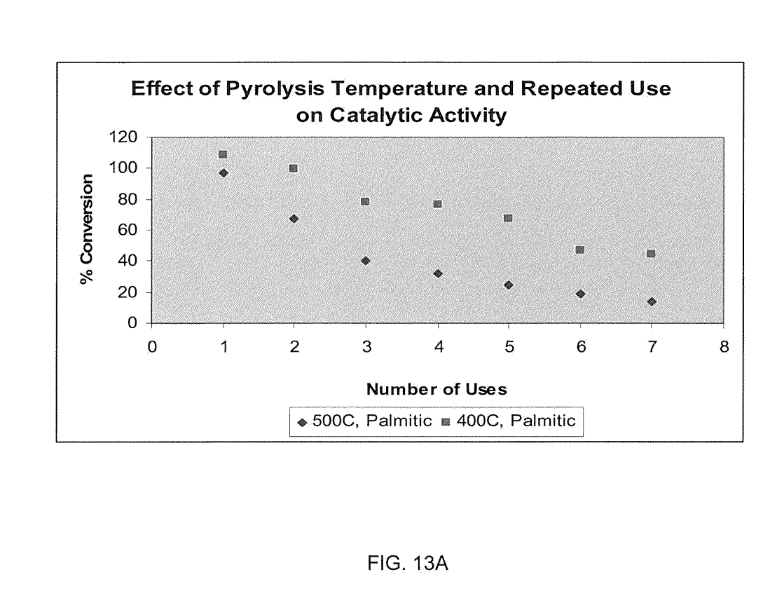

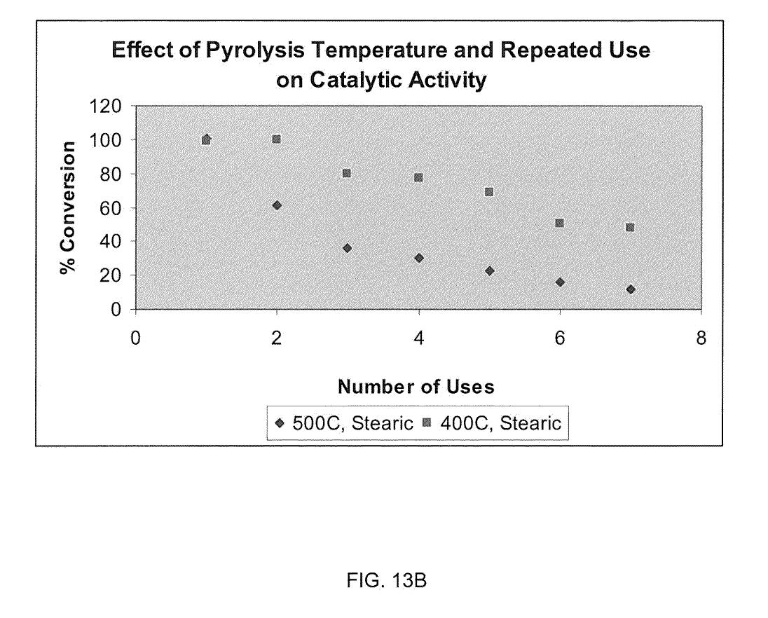

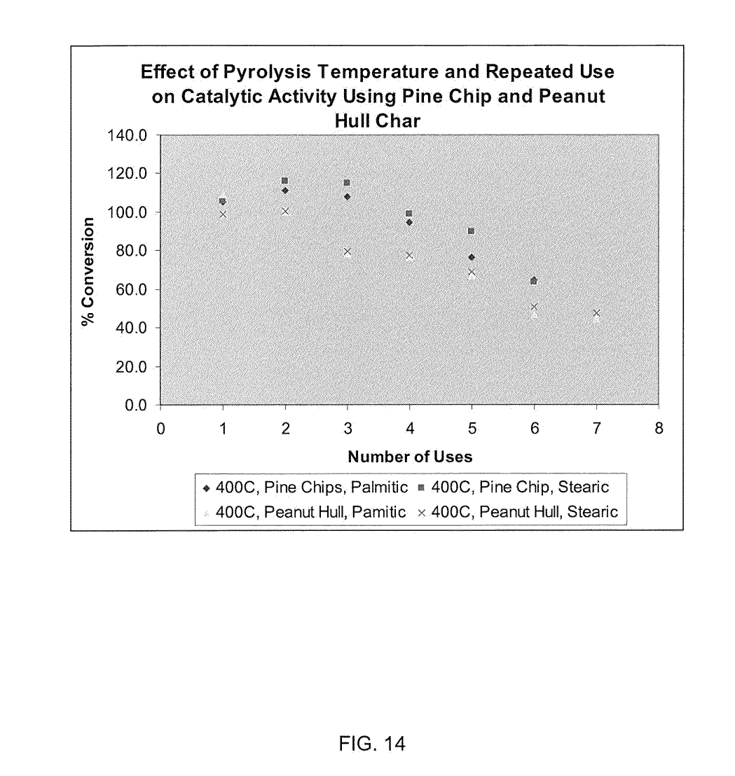

Esterification Catalytic Activity: All sulfonated chars were demonstrated to be catalytically active for esterification of palmitic and stearic acid with methanol (FIGS. 10, 11, 12; Tables 4, 5). Chars treated with ozone only were not active and sulfonation of these chars did not increase esterification activity relative to chars that were directly sulfonated (data not shown). Catalytic activity appeared to follow the trends implied by the DRIFT analysis of the synthesized catalysts. Chars synthesized at lower temperatures and sulfonated at lower temperatures (400° C. for pyrolysis, 100° C. for sulfonation) had the highest activity (FIG. 13). Additionally, sulfonated char generated from pine chips appeared to have the highest activity relative to peanut hull char (FIG. 14). Repeated reuse of the catalysts indicated a reduction in activity, but not a complete loss in activity (FIGS. 13, 14). DRIFT analysis of the reused char did indicate a loss in the peaks representative of —SO₃ functional groups (FIG. 9), but it is not clear from these data whether this was due to leaching of the active sites or adsorption of poisons on the surface of the catalyst.

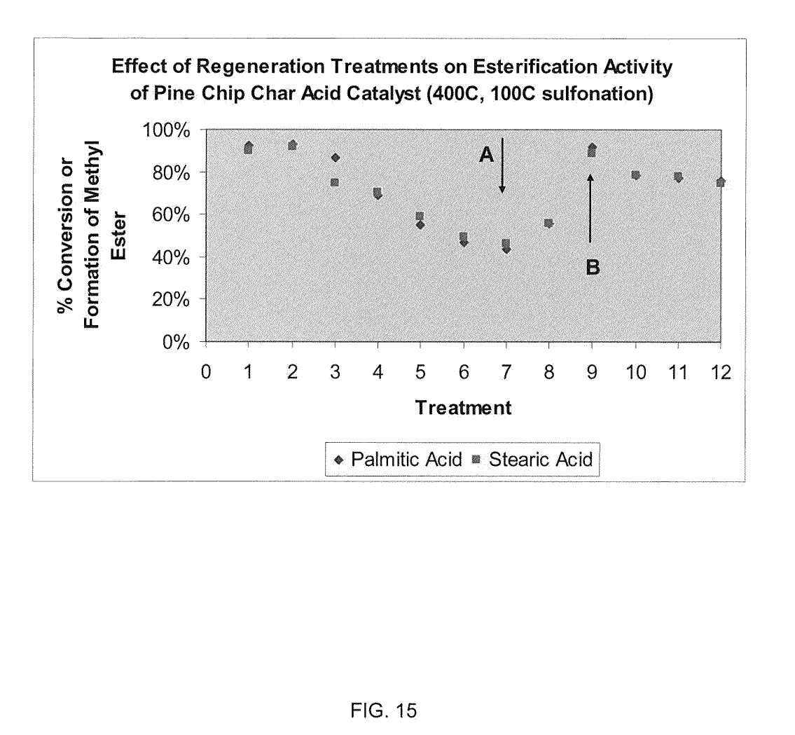

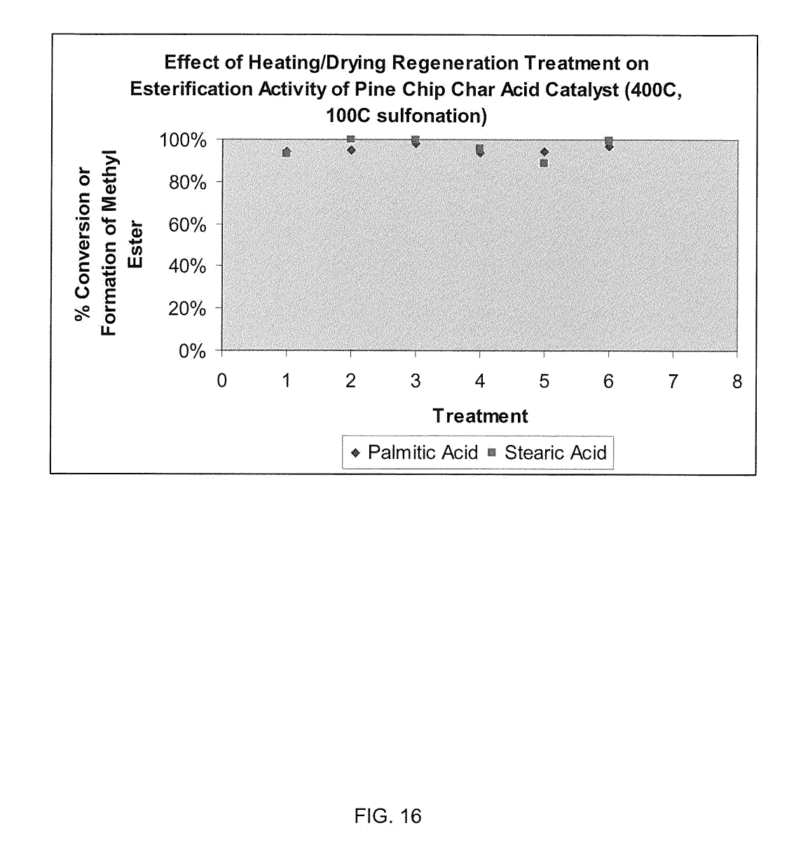

Catalyst Regeneration and Reuse: Since catalytic activity declined with repeated use of the catalyst (FIG. 13, 14), different regeneration techniques were explored to determine if activity could be regenerated. Clearly, rinsing the char catalyst with methanol in between batch reactions was not effective, since this resulted in declining activity (FIG. 13, 14). Subsequently, contacting the char catalyst with heptane and then drying at 125° C. or 250° C. was determined to significantly increase catalytic activity of the char catalyst (FIG. 15). Finally, just simply drying the char catalyst at 125° C. (1 h), without solvent contact, resulted in a consistent, high catalytic activity with repeated uses of the char catalyst (FIG. 16).

### Conclusions:

Low temperature pyrolysis of biomass (<600° C.) generated a char (i.e., a carbon backbone) that is conducive to sulfonation and formation of acid functional groups on the carbon surface.

Chars generated at 400° C. and sulfonated at 100° C. were highly active, solid acid carbon supported catalysts.

Drying the solid acid carbon supported catalyst at 100° C. for 1 h maintained esterification catalytic activity.

Solid acid catalysts can be generated from biomass and renewable carbon sources without the need to refine the biomass, contrary to previous work on the generation of solid acid catalysts from refined sugars or carbohydrates (e.g., glucose, starch, or cellulose) and glucose impregnated petroleum derived polymers.

TABLE 1
Physical and chemical characteristics of char used to develop catalysts.

| | | | | | Wood Chip | |
|---|---|---|---|---|---|---|
| | | | | | Char | |
| | Activated | Peanut | Peanut Hull | Palm Oil | (Mean ± | Poultry Litter |
| Materials | Carbon | Hull Char | Char | Char | SD) | Char |
| Properties | (Lignite Coal) | (472° C.) | (500° C.) | (500° C.) | 400° C. | 500° C. |
| Generation | Steam | Pyrolysis- | Pyrolysis- | Pyrolysis- | Pyrolysis-N₂ | Pyrolysis-N₂ |
| Process | | N₂/Steam | N₂ | N₂ | | |
| Reactor/Residence | Unknown | Continuous/ | Batch/40 min | Batch/40 min | Batch/40 min | Batch/40 min |
| Time | | 10 min | | | | |
| pH | 4.6 | 10.1 | 10.5 | 5.9 | 7.83* | 9.9 |
| Bulk | ND | ND | 0.61-0.72 | ND | ND | ND |
| Density, g | | | | | | |
| cm⁻³ | | | | | | |
| Carbon, % | ND | 73 | 64-68 | ND | ND | 46.3 ± 2 |
| (dry basis) | | | | | | |
| Nitrogen, % | ND | 2 | ND | ND | ND | 3.7 ± 0.2 |
| Surface | 506 ± 34 | 1.54 ± 0.52 | 3.2 ± 0.6 | 1.0 ± 1.46 | 3.89 ± 0.14 | 6 ± 2 |
| Area, m² g⁻¹ | 494 ± 57 | 0.52 ± 0.31 | NP | 0.12 ± 0.30 | NP | NP |
| O₃ treated, | | | | | | |
| 30 min | | | | | | |
| Pore | 0.55 ± 0.005 | 0.003 ± 0.001 | 0.004 ± 3e⁻⁴ | ND | ND | NP |
| Volume (ml | | | | | | |
| g⁻¹) | | | | | | |
| Selected | | | | | | |
| Elements | | | | | | |
| (ppm or mg | | | | | | |
| kg⁻¹) | Mean | Mean | Mean | Mean | Mean | Mean |
| Co | ND | ND | 0.67 | ND | ND | ND |
| Cu | ND | 18.5 | 17.9 | 46.1 | ND | ND |
| Mn | ND | 142 | 157.3 | 34.0 | ND | ND |
| Mo | ND | 10 | ND | <1 | ND | ND |
| Ni | ND | BDL | 3.95 | <2 | ND | ND |
| Fe | ND | 1,294 | 1094.4 | 276.1 | ND | ND |
| Ca | ND | 5,414 | 4,090.4 | 1,182.0 | ND | ND |
| K | ND | 19,311 | 20,700.2 | 4,554.0 | ND | ND |
| Mg | ND | 2,716 | 2,478.2 | 1096.1 | ND | ND |

ND—Not Determined,
NP—Not Performed,
AC—Activated Carbon,
BDL—Below detection limit of 2 ppm
*Measured by Down to Earth Energy

TABLE 2
Particle size analysis of adsorbents used in the
ammonia adsorption studies.

| | Activated | Palm Oil | Peanut Hull | Poultry Litter |
|---|---|---|---|---|
| | Carbon^{a} | Shell Char | Char | Char |
| Sieve/Adsorbent | (%, w/w) | (%, w/w) | (%, w/w) | (%, w/w) |
| 4 (4.75 mm) | 5 | 0.00 | 0.00 | 9.03 |
| 6 (3.35 mm) | NP | 2.24 | 32.32 | 26.16 |
| 7 (2.80 mm) | NP | 13.06 | 11.92 | 13.19 |
| 8 (2.36 mm) | NP | 45.15 | 6.87 | 34.03 |
| 10 (2.00 mm) | 5 | 9.33 | 10.71 | 17.59 |
| >12 (1.70 mm) | NP | 30.22 | 38.18 | 9.03 |

^{a}, taken from Norit Americas website (http://www.noritamericas.com/1.6.cfm?type=TDS)
NP, not performed

TABLE 3
Literature analysis of FTIR analysis of functionalized carbon and peak
assignments.

| Functional | | Wavenumber | Biomass or | | | |
|---|---|---|---|---|---|---|
| Group | Description | (cm⁻¹) | Material | Process | Application | Reference |
| —OH | Phenolic | 3546 | Phenol- | Pyrolysis | Adsorption | A |
| | hydroxyls | | formaldehyde | (350-600° C.) | | |
| | | | resins | 99% H₂SO₄, | | |
| | | | | 3 h, 160-250° C. | | |
| C═C | Stretching, | 1600 | Phenol- | Pyrolysis | Adsorption | A |
| | polycyclic | | formaldehyde | (350-600° C.) | | |
| | aromatics | | resins | 99% H₂SO₄, | | |
| | | | | 3 h, 160-250° C. | | |
| C═O | Carbonyl | 1683 | Phenol- | Pyrolysis | ″ | A |
| | groups | 1709 | formaldehyde | (350-600° C.) | | |
| | | | resins | 99% H₂SO₄, | | |
| | | | | 3 h, 160-250° C. | | |
| C═O | Carbonyl | 1713 | Bambo | 80% H₂SO₄, | Catalytic organic | A, B, C |
| | stretching | 1740 | | 3 h, 80° C., | synthesis | |
| | carboxylic | | | washed, 20% | | |
| | acid groups | | | H₂SO₄, 2 h, | | |
| | | | | 80° C., washed | | |
| S═O | Sulfonic | 1035 | Cork | Pyrolysis | Cation Exhange | B, C |
| | Acid Group | 1037 | | (300-600° C.), | | |
| | | 1228 | | 3 h, 25% | | |
| | | | | H₂SO₄, 1.5 h, | | |
| | | | | 80-120° C. | | |
| —SO₃ | Sulfonic | 600 | Cork | Pyrolysis | Cation Exhange | B, C |
| | Acid Group | 1040 | | (300-600° C.), | | |
| | | 1200 | | 3 h, 25% | | |
| | | | | H₂SO₄, 1.5 h, | | |
| | | | | 80-120° C. | | |
| C—S | Sulfonic | 605 | Bambo | 80% H₂SO₄, | Catalytic organic | B |
| | Acid Group | 645 | | 3 h, 80° C., | synthesis | |
| | | | | washed, 20% | | |
| | | | | H₂SO₄, 2 h, | | |
| | | | | 80° C., washed | | |

A, Carbon 40 (2002) 2323-2332;
B, Catalysis Communications 9 (2008), 1579-1582;
C, Bioresource 44 (1993) 229-233

TABLE 4
Change in percent conversion of FFA's during esterification
reactions catalyzed by granular and powdered ozonated and
sulfonated pine pellet char.

| | Sample | | | |
|---|---|---|---|---|
| | % Conversion Granular | | % Conversion Powder | |
| Reaction Time in | Methyl | Methyl | Methyl | Methyl |
| Minutes | Palmitate | Stearate | Palmitate | Stearate |
| 0 | 1% | 1% | 1% | 0% |
| 40 | 35% | 23% | 24% | 25% |
| 80 | 65% | 42% | 54% | 48% |
| 120 | 84% | 55% | 74% | 63% |
| 160 | 93% | 62% | 88% | 75% |
| 200 | 100% | 66% | 96% | 81% |
| 240 | 103% | 69% | 100% | 88% |

TABLE 5
Change in percent conversion of FFA's during esterification
reactions catalyzed by granular and powdered ozonated and
sulfonated peanut hull char.

| Sample | |
|---|---|
| % Conversion | % Conversion |
| Granular | Powder |

| Reaction Time | Methyl | Methyl | Methyl | Methyl |
|---|---|---|---|---|
| in Minutes | Palmitate | Stearate | Palmitate | Stearate |
| 0 | 2% | 1% | 1% | 1% |
| 40 | 39% | 32% | 49% | 44% |
| 80 | 72% | 60% | 80% | 74% |
| 120 | 93% | 77% | 93% | 87% |
| 160 | 96% | 82% | 100% | 94% |
| 200 | 101% | 87% | 104% | 97% |
| 240 | 104% | 87% | 105% | 98% |

TABLE 6
Compositional analysis of biomass feedstocks used to
generation solid acid catalyst via low temperature pyrolysis
coupled with sulfonation.

| | Materials | | |
|---|---|---|---|
| | Pelletized | | Pine Chip |
| Properties | Peanut Hulls | Pine Pellets | Char |
| Cellulose, % (dry basis) | 43 ± 0.03 | 47.3 ± 0.5 | 51.7 ± 0.9 |
| Hemicellulose, % (dry basis) | 4.0 ± 0.2 | 14.4 ± 1.5 | 18.7 ± 1.3 |
| Lignin, % (dry basis) | 29.2 ± 0.25 | 24.6 ± 0.42 | 28.2 ± 0.3 |
| Ash, % (dry basis) | 2.6 | 0.14 | 0.30 |
| Bulk Density, kg/m³ | 609-720 | ND | ND |

ND, not determined

### Example 2

Reusable solid acid carbon supported catalysts were generated from biomass by pyrolyzing peanut hull pellets, wood chips, and pine pellets at 400 and 500° C. to generate a soft to hard carbon backbone (i.e., biochar) for addition of acidic functional groups. Acid catalysts were synthesized by sulfonating the biochar using concentrated H₂SO₄ at 100, 150 and 200° C. (12 h). Sulfonated biochars esterified fatty acids within 30-60 minutes at 55-60° C. with ˜90-100% conversion. Drying the acid catalysts for 1 hour at 125° C. between uses maintained esterification activity and allowed reuse.

### Introduction

The objectives of this research were to develop reusable and recoverable solid, carbon supported, porous acid catalysts for biodiesel generation using biochar generated from lignocellulosics. It was theorized that low temperature (400-600° C.), slow pyrolysis of biomass would generate a highly cross-linked, multi-ringed, aromatic structure anchored to lignin that could be easily functionalized with catalytically active acidic groups. Based on previous analysis of char generated by pyrolysis of pure biomass components (cellulose, hemicelluloses, and lignin) and whole biomass, it is theorized that lignin would undergo partial decomposition and hemicellulose and cellulose would undergo a series of thermal homolysis, hydrolysis, dehydration, and molecular rearrangement reactions to form a polymerized aromatic structure. Catalysts derived from biochar, contrary to refined carbohydrates, zeolites, and resins would be environmentally friendly (e.g., separation of glucose from biomass, mining required for zeolites, or use of petroleum feedstocks for synthesis of resins would not be required).

### EXPERIMENTAL

### Biomass Sources and Biochar Generation

Pelletized peanut hulls and pine logging residues, and wood chips, were used to generate biochar. Pelletized peanut hulls were supplied by Golden Peanut Co. LLC. Pelletized pine pellets and wood chips were purchased from local suppliers. The pelletized peanut hulls were typically 15 mm in length and 8 mm in diameter and the pine chips 4-18 mm in length, 15-30 mm in width, and 1-5 mm in thickness. The composition of biomass materials (cellulose, hemicelluloses, and lignin) were analyzed in labs at UGA and reported in Table 6.

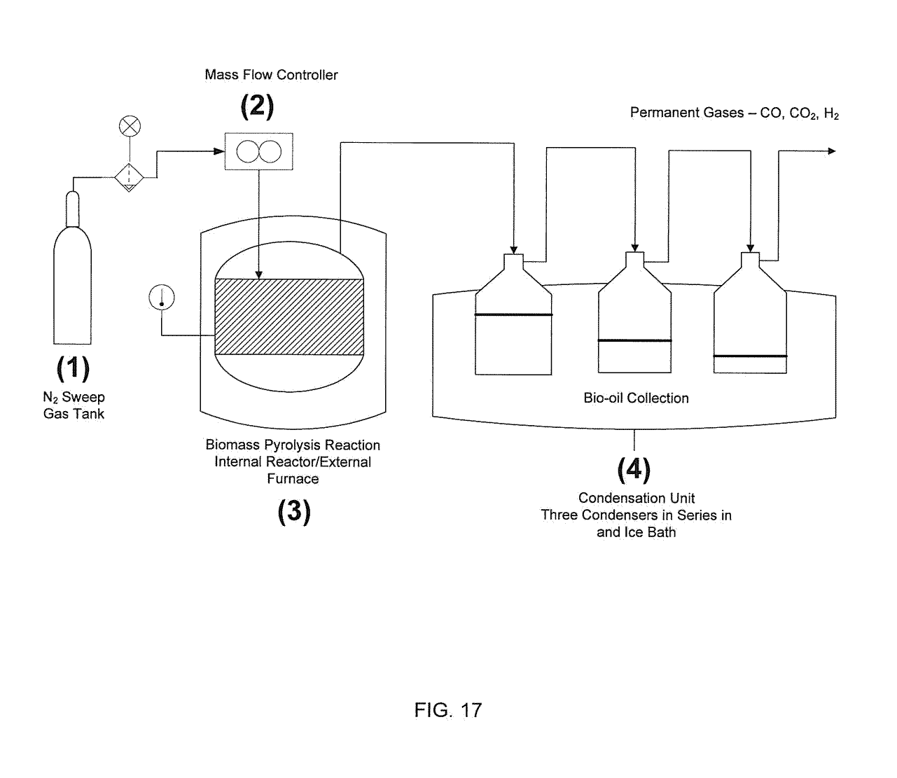

The pyrolysis unit consisted of a batch reactor (316SS, 23×23×23 cm reactor, with a N₂ purge line and exhaust) located inside a furnace (Thermolyne, Barnstead Inc, La; Thermolyne single set point, 1200° C. max, 10° C./min ramp) followed by a condensation unit (FIG. 17). Pure nitrogen was used as an inert sweep gas (½″ stainless steel tubing) and controlled using a mass flow controller. A thermowell located 2 inches from the bottom of the reactor within the biomass was used to monitor and manually control temperature (type K thermocouple connected to a CR23X datalogger, averaging at 2 min intervals). In this experiment, char was prepared over a range of temperatures, including 400, 500, and 600° C. and held for 40 min in the pyrolysis reactor with a N₂ flow at 1-2 L min⁻¹. Initial mass loading of the pyrolysis reactor depended on biomass type and ranged from 2-5 kg for pine chips and 2-3 kg for pelletized peanut hulls and pine.

### Ozone Treatment

In some cases, char was exposed to ozone in a fixed-bed reactor (1 in ID×12 in L, FIG. 5) packed with a defined mass of the char (25 g). Ozone in air was passed downward across the carbon at 6.1 L min⁻¹ (total flow) and 33 g m⁻³ O₃ for a period of 6 hours (23° C.). An ozone generator (OL100H/DS, Yanco Industries Ltd., B.C., Canada), utilizing a high frequency corona discharge, was used with a medium grade tank of oxygen (99.9%, National Wielders, N.C.) to generate the ozone required for these experiments.

Catalyst Generation

Next, the char generated above was further reacted to attach or generate acidic functional groups on the carbon surface. Acid catalysts included a weak acid functionalized carbon (char treated with ozone only) and a strong acid, —SO₃H functionalized char. For the weak acid catalyst, the char was oxidized using ozone at room temperature in a packed-bed reactor. We anticipate that this process introduced a high density of acidic groups (e.g., carboxylic acids, phenolic, and lactonic groups) throughout the carbon pores—this conclusion is based on our recent NH₃ adsorption studies of ozonated char and literature analysis (Journal of Hazardous Materials. 2009, 164, 1420-1427).

For development of a strong acid catalyst (—SO₃H), the chars (400, 500, and 600° C.) were sulfonated based on analysis of a range of methods described in the literature (Yantasee et al., 2004, Toda et al., 2005). First, the pelletized chars were ground into smaller particles using a mortar and pestle (pine chip char was used as generated). Pellets were ground and then sieved on 4-12 mesh screen sieves, retaining all char particles between in that specific range. The sieved char was then weighed (25 g) and placed in an ACE glass reactor with glass wool and mesh screen “plug” to keep the char in place. Ozonation of the char was accomplished as noted above.

Subsequently, 12.5 grams of the previously ozonated char or un-ozonated char, was placed in a beaker, and contacted with 20 mL of concentrated sulfuric acid (99% H₂SO₄). The acid was mixed with the char via periodic stirring (15 min), and then excess acid was decanted. The residual wet solids (char plus acid) were then transferred to a ceramic crucible, placed in muffle furnace, and heated for 12-18 hours at 100, 150, or 250° C. After heating, the char was cooled and rinsed 2-10× with 50-100 mL of deionized water. The pH of the rinsate was measured after each washing and the char was then dried in an oven at 110° C. overnight.

### Catalyst Characterization

The physical and chemical characteristics of the chars, including pH, surface area, bulk density, and the elemental composition were previously determined (Journal of Hazardous Materials. 2009, 164, 1420-1427, which is herein incorporated by reference for the corresponding discussion). Moisture, volatiles and ash content in the biomass, biochar, and treated biochars (i.e., catalysts) was determined by ASTM D5142 using a proximate analyzer (LECO Model TGA701). Ultimate analysis (elemental C, H, N, S, and O (by difference) in % (w/w)) was performed in an ultimate analyzer (LECO, model CHNS-932) following ASTM D3176.

To qualitatively assess the formation of functional groups on the surface of the treated char, FTIR analysis (in DRIFT mode) was performed on the base chars (i.e., just pyrolyzed and not functionalized) and the sulfonated chars. The chars were partially crushed and analyzed directly using a Diffuse Reflectance Infrared Fourier Transform Spectrometer or DRIFT cell (Varian 2000 FT-IR spectrometer, Scimitar Series). All generated catalysts and base chars were analyzed in triplicate. The DRIFT cell sample cup was completely packed with particles of the char and sulfonated char and leveled before analysis (the particles were not crushed to a fine powder and KBr was not used). The chars were not ground to fine powder and mixed with KBr because previous analysis indicated that this procedure can eliminate signals due to dilution of key functional groups on the surface of treated material, especially for granular activated carbon or similar materials (Chem. Mater. 1997, 9, 176-183, which is herein incorporated by reference for the corresponding discussion).

For each analysis, a base char (i.e., non-functionalized) was used to generate a reference spectrum and subtracted from the FTIR spectrum of the sulfonated chars. The base char and sulfonated chars (˜20 mg) were scanned from 600 to 3500 wavenumbers (cm⁻¹) at a total of 64 scans and a resolution of 4 cm⁻¹. The DRIFT spectrum was output in the Kubelka-Munk form.

Grazing angle attenuated total reflection Fourier transform infrared spectroscopy (GATR-FTIR) was also used to provide information on surface functional groups. ATR was used to deconvolute the functional groups on the surface from the bulk phase by using an incident angle with a limited depth of penetration (˜150 nm). Our experimental setup allowed for a variable angle, so that the incident angle could be optimized for highest sensitivity with different types of samples.

### Esterification Activity

Acid Catalyst Screening

Each acid catalyst was tested for its ability to form methyl esters using free fatty acids (palmitic and stearic acids) and methanol. The catalysts were tested in a small scale batch reactor system (Reacti-Therm, Pierce—Thermo Scientific). The reactor was pre-calibrated to determine temperature output versus setting input and temperature was monitored using a thermocouple. Pure palmitic and stearic acids (99+%) were used as model free fatty acids and purchased for use (Sigma Aldrich).

A known amount of catalyst (typically 0.2 g) was charged into a vial (5 mL total volume) with a known initial volume of palmitic (C₁₆-saturated FFA) or stearic (C₁₈-saturated, both at 200 ppm) and methanol (4 mL, excess methanol). The mixture was then heated at 55-60° C. and sub-samples taken as function of time to determine the formation of methyl esters. Control reactions consisted of the untreated char (negative control) or use of HCl (positive control). The liquid sub-samples were analyzed and quantified using a GC/FID or GC/MS and hexadecane was used as an internal standard to determine the concentration of methyl esters formed. Fractional removal or % conversion of palmitic or stearic acid were based on the defined initial concentrations of the FFAs (maximum theoretical amount of methyl ester that could form) and the concentration of the methyl esters of the FFAs that formed during the catalytic reaction, thus % X or Conversion=[C_{final}(FFA-ME)]/Cᵢₙᵢₜᵢₐₗ(FFA-ME)×100, where C is concentration, FFA is free fatty acid, and FFA-ME is fatty acid methyl ester.

Acid Catalyst Testing Using Simulated Feedstocks

The acid catalyst identified as having high esterification activity with palmitic and stearic acid (400° C. pine chip char, sulfonated at 100° C.) was then tested for its activity using soybean oil (Kroger Brand Pure Vegetable Oil) and rendered poultry fat (Fieldale Farms Poultry, LLC Baldwin, Ga. 30511; % FFA of 2.53±0.11 and moisture content of 0.16±0.05 [%]). Catalytic reactions were performed in a 200 mL, three-neck round-bottom flask (24/40, LabGlass with a thermometer adaptor and RTD probe) connected to a water-cooled condenser (Liebig Condenser) and an electronic heater/stirrer (VWR model # VMS C7). The round-bottom flask (batch reactor) was immersed in a water bath and the temperature was maintained at 64-65° C. with the RTP probe. The reaction mixture was rapidly stirred using a magnetic stirring bar (500-800 rpm). Palmitic (1.9 or 2.2 g) and stearic (0.91 or 1.82 g) acid were added to poultry fat (18 or 36 g) to generate—10-13.5% FFAs in the fat. This mixture was heated to 60° C. and agitated to generate a clear solution and the temperature then raised to 65° C. The solid acid catalyst (2 g) was then mixed with methanol to excess (16.5 mL for 18 g of fat or 33 mL for 36 g of fat) and transferred to the batch reactor where the esterification reaction was allowed to proceed for 2 hours at 65° C. under agitation. After cooling the mixture was filtered into a graduated cylinder and allowed to separate into two layers—a top layer (methanol) and bottom layer (fat, FFA-methyl esters). Both layers were analyzed by gas chromatography using ASTM Method D6584-07 and by potentiometric titration for total acids using ASTM Method 664-07. When calculating % conversion using the ASTM Method 664, equation 1 was used and based on the measured total acid number (TAN).

$$\begin{matrix}Conversion ( \% ) = \frac{Initial acid number - final acid number}{Initial acid number} \times 100 , & ( 1 ) \\ Where , the acid number ( mg KOH \text{/} g ) = \frac{( A - B ) \times M \times 56.1}{W} , & ( 2 )\end{matrix}$$

and M is the concentration of the KOH solution (mol L⁻¹); A the volume of KOH standard solution used in the titration (mL); B the volume of KOH standard solution used in the titration of the blank (mL); 56.1 the molecular weight of KOH (mol g⁻¹); and W the sample weight (g).

In a similar experiment, a solid acid catalyst (400° C. pine chip char, sulfonated at 100° C.) was tested for its capability of esterifying palmitic and stearic acid in soybean oil. Palmitic (1.9 g) and stearic (0.91 g) acid were added to soybean oil (18 g or 19 mL) to generate ˜13.5% FFAs in the oil. This mixture was heated to 60° C. and agitated to generate a clear solution and the temperature then raised to 65° C. The solid acid catalyst (2 g) was then mixed with methanol to excess (16.5 mL) and transferred to the batch reactor where the esterification reaction was allowed to proceed for 2 hours at 65° C. under agitation.

### Analytical Methods

Standards and Quality Control

Standard stock solutions were prepared from certified analytical reference materials (Matreya Company, Pleasant Gap, Pa.) which included, n-hexadecane, methyl pentadecanoate, methyl hexadecanoate (palmitic acid, methyl ester), methyl octadecanoate (stearic acid, methyl ester), methyl heneicosanoate, hexadecanoic acid, and octadecanoic acid (10,000 ppm, dissolved in a 1:1 mixture of dichloromethane and heptane). Each standard was analyzed in triplicate to determine standard deviations (SD) and relative standard deviations (RSD) of response factors.

Solid Acid Catalyst Reactions with Palmitic and Stearic Acids

Standard curves for methyl hexadecanoate (palmitic acid, methyl ester) and methyl octadecanoate (stearic acid, methyl ester), using n-hexadecane as a quantitative internal standard, were prepared and used to quantify palmitic and stearic acid methyl esters. Conversion of palmitic and stearic acid to methyl esters were quantified using GC/FID or GC/MS methods.

Methyl ester concentrations were determined using the GC/FID with direct on-column 1 uL injections under conditions specified in ASTM Method D6584-07. An SRI Model 610 gas chromatograph equipped with an FID detector and capillary column (Restek MXT widebore metal column, 15 m) was used with the following temperature program −50° C. for 1 min, ramped to 180 C at 15° C./min, ramped to 230° C. at 7° C./min, ramped to 380° C. at 30° C./min and held 10 min.

In some cases methyl hexadecanoate and methyl octadecanoate concentrations were determined using GC/MS. An Agilent 6890 chromatograph operated with split (50:1) and equipped with a MS detector (Agilent 5973) and capillary column (HP-5, 0.25 mm I.D., 0.25 μm film thickness, 30 m) was used, with the following conditions and temperature program—solvent delay of 2.8 min, inlet 230° C., detector 280° C. (MS interface temperature), 1 mL min⁻¹ He, 50° C. for 1 min followed by a ramp at 25° C. min⁻¹ to 200° C., and then a ramp at 3° C. min⁻¹ to 240° C. and held for 15 min. Masses were scanned from 30-500 mass units and the injection volume was 1 μL. The methyl esters were identified based on retention times with standards and mass spectral analysis (MSD ChemStation D.03.00.611 and NIST 98 database). Quantification was made using n-hexadecane as an external standard (500 ppm) and single concentration standards of methyl hexadecanoate and methyl octadecanoate (500 ppm).

Solid Acid Catalyst Reactions with Rendered Poultry Fat

Conversion of palmitic and stearic acid to methyl esters in poultry fat were quantified using GC/FID and ASTM Method D6584-07 and by potentiometric titration using ASTM Method D664-07 with a KEM Model AT-610 automatic potentiometric titrator.

In the GC method a 100 pt sample of fat (original, spiked, and reacted samples) was prepared for gas chromatographic analysis by adding 100 uL of tricaprin standard (internal standard for quantifying glycerides) and 100 μL of N-methyl-N-trimethylsilyltrifluoracetamide (MSTFA) to derivatize the glycerides. After 25 minutes at room temperature, 8 mL of n-heptane was added and to 2.0 mL aliquots of the derivatized solutions was added 100 μL of 10,000 ppm solutions of n-hexadecane, methyl pentadecanoate and methyl heneicosonate standards to make a 500 ppm internal standard for quantifying methyl palmitate and methyl stearate. The samples were analyzed using conditions listed for oil analysis by ASTM D6584-07.

Free fatty acid levels (original, spiked, and reacted samples) using potentiometric titrations were performed in the following manner using ASTM 664. Samples (−20 g of unfortified control sample oils or ˜2 g of biodiesel reaction products) were dissolved in a mixture of 50% toluene and 49.5% 2-propanol containing 0.5% water and titrated potentiometrically using a solution of 0.1 N potassium hydroxide in 2-propanol (the electrode was a glass combination electrode with a saturated lithium chloride solution). The meter readings are plotted automatically against the respective volumes of titrating solution and the end points are recorded at well-defined inflections in the resulting curve. The Total Acid Number (TAN) was determined based on equation 2.

Solid Acid Catalyst Reactions with Spiked Soybean Oil

Conversion of palmitic and stearic acid to methyl esters in soybean oil were quantified using the methods as discussed above.

### Results and Discussion

Char Characterization

Significant differences in biomass composition, which ultimately may have affected char structure, were observed. Peanut hulls had a significantly higher ash content and lower hemicellulose concentration compared to pine pellets and chips (Table 6). The high ash content of the peanut hulls is reflected in high levels of calcium and potassium in the biochars (Ca, 4000-4600 mg L⁻¹; K, 15,000-20,000 mg L⁻¹). Chars formed at low temperature (<600° C.) and relatively short holding times (<1 h) typically have low surface area. The generated chars had surface areas ranging from 2-4 m²/g, much lower than typical activated carbon (500-1,500 m²/g). These surface areas are similar to chars previously used to synthesize solid acid catalysts and generated from cellulose, sucrose, and glucose via low temperature pyrolysis (4-7 m²/g, Lou et al., 2008). However, it must be pointed out that high surface area is not a prerequisite for successful catalysis, especially if the process of interest occurs under diffusion limited conditions. In those cases much of the surface area is not utilized and the cost and engineering effort to generate the higher surface is futile.

DRIFT and ATR Analysis

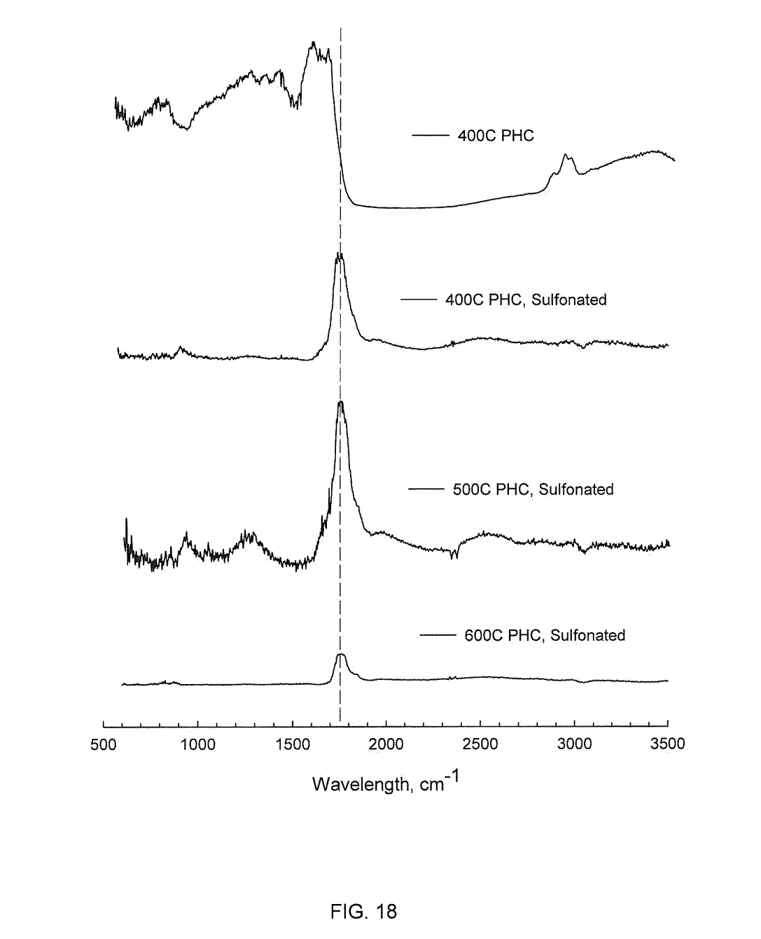

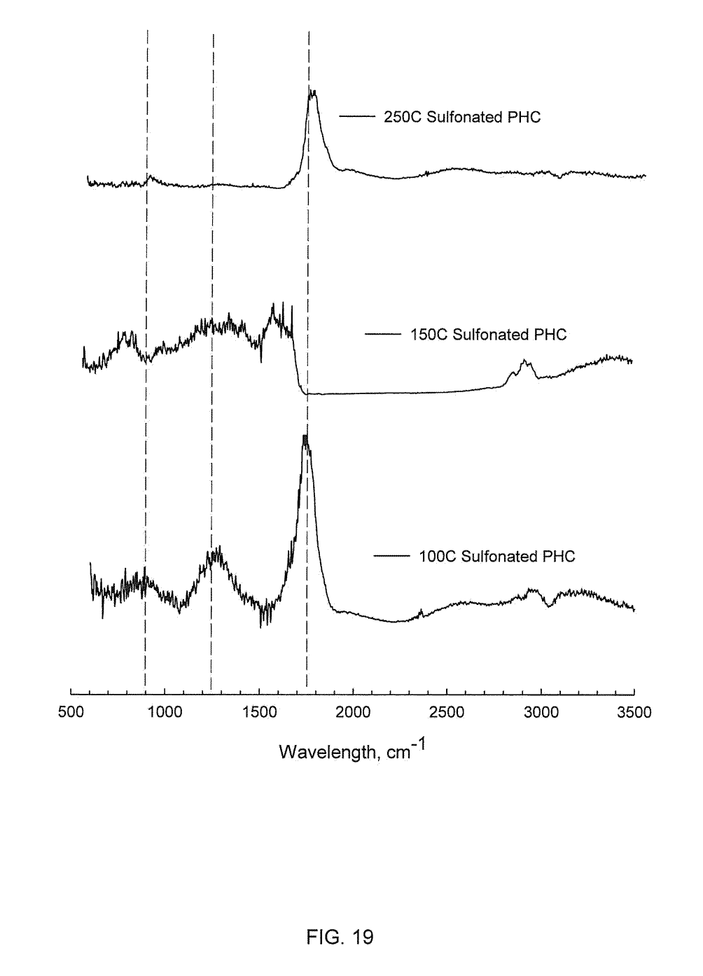

In general, based on DRIFT analysis, it appeared that lower pyrolysis temperatures increased the reactivity of the chars towards sulfonation and lower sulfonation temperatures prevented over oxidation of the char and increased the formation of —SO₃ groups. A large peak at 1750 cm⁻¹, indicative of a carboxylic acid group (Table 3) appeared in the sulfonated peanut hull chars generated at 400 and 500° C., and a much smaller peak was generated at 600° C. (sulfonation temperature −250° C.; FIG. 18). Peaks suggestive of —SO₃ formation did not appear unless the pyrolysis and sulfonation temperature was reduced. A reduction in pyrolysis temperature from 600 to 500 or 400° C. and a sulfonation temperature from 250 to 150° C. and 100° C. qualitatively appeared to increase the peaks indicative of a sulfonic acid group in the peanut hull and pine chip chars (FIGS. 18 and 19). Increasing sulfonation efficiency with decreasing activation temperature was also confirmed by measuring the sulfur content of the treated biochar (one common batch of biochar) and estimating —SO₃H density (Table 7).

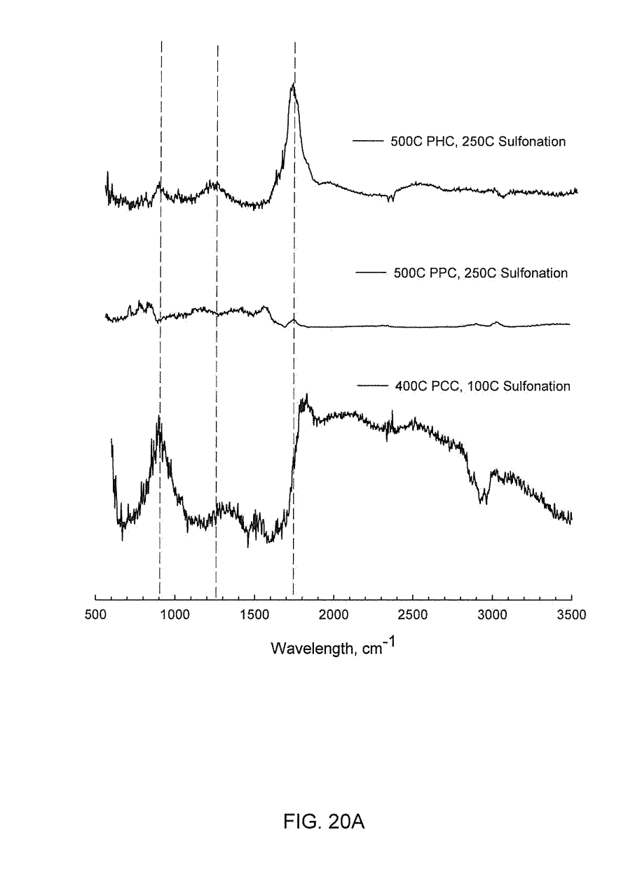

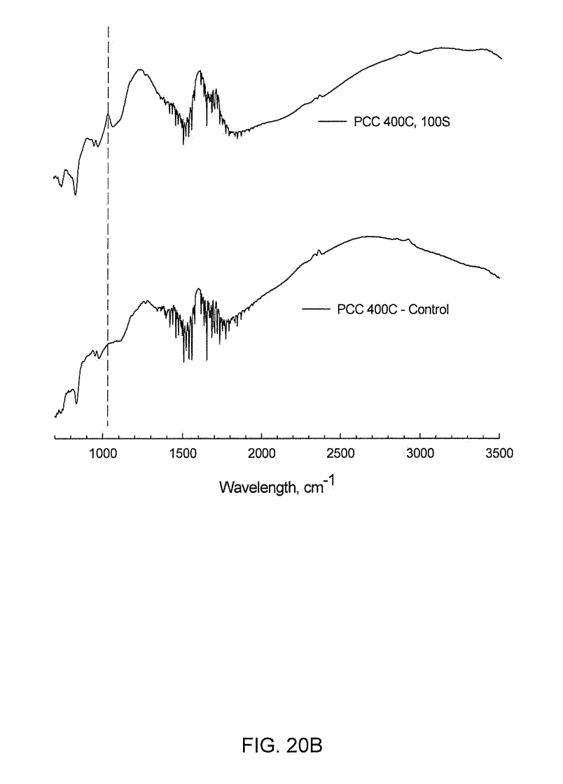

At a pyrolysis temperature of 400° C. and a sulfonation temperature of 100° C. the peak at 1750 cm⁻¹ was reduced and broader less distinctive peaks at 1550-1600 cm⁻¹, 1100-1500 cm⁻¹, and 700-900 cm⁻¹ appeared. It has been suggested that peaks at 1037, 1040, and 1200 cm⁻¹ are indicative of S═O and —SO₃ groups (Table 8). The formation of peaks most indicative of —SO₃ groups occurred when pine wood chips (i.e., not pelletized) pyrolyzed at 400° C. were sulfonated at 100° C. (FIG. 20). ATR analysis of the sulfonated char also confirmed the formation of a sulfonic acid functional group on the biochar surface; pyrolyzed pine chip char (400° C.) when sulfonated at 100° C. formed a distinct peak at 1050-1100 cm⁻¹ (FIG. 20). Our results are similar to reports in the literature on the sulfonation of pyrolyzed cork and phenolic fiber Kynol™ for synthesis of ion-exchange materials; as pyrolysis temperature is increased the degree of sulfonation of the chars decreases for a fixed sulfonation condition [300-400° C. optimum for the cork (300-600° C.) and 525° C. optimum for the fibers (525-600° C.)—Bioresource Technology. 1993, 44, 229-33; Carbon. 2002 40, 2323-2332, which are herein incorporated by reference for the corresponding discussion].

Esterification Catalytic Activity

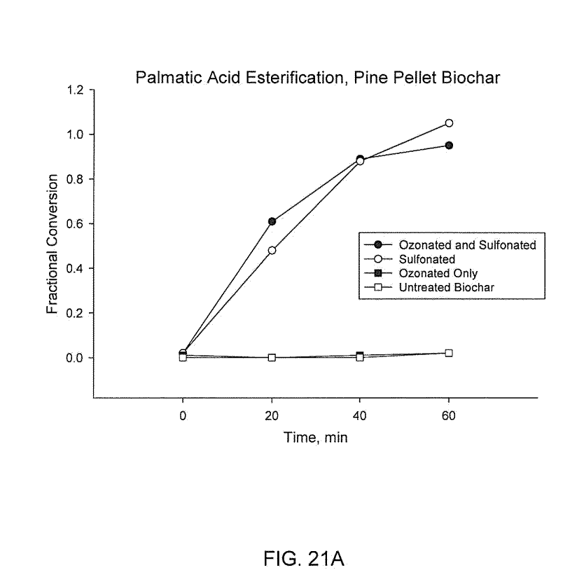

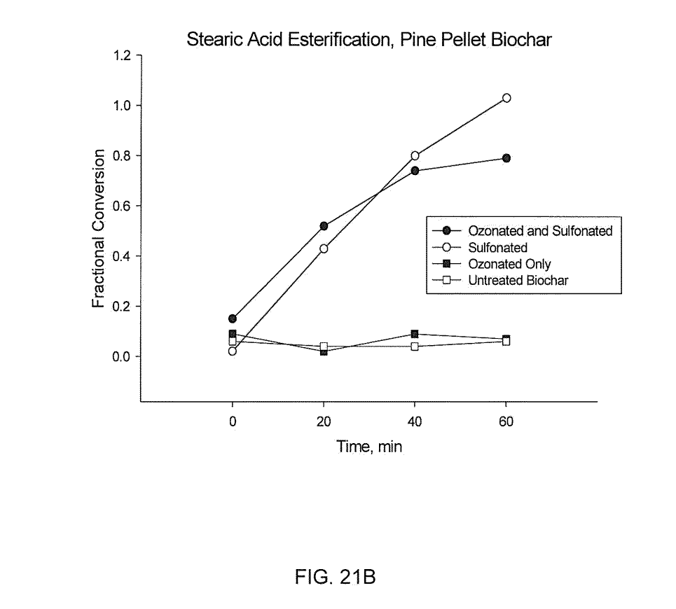

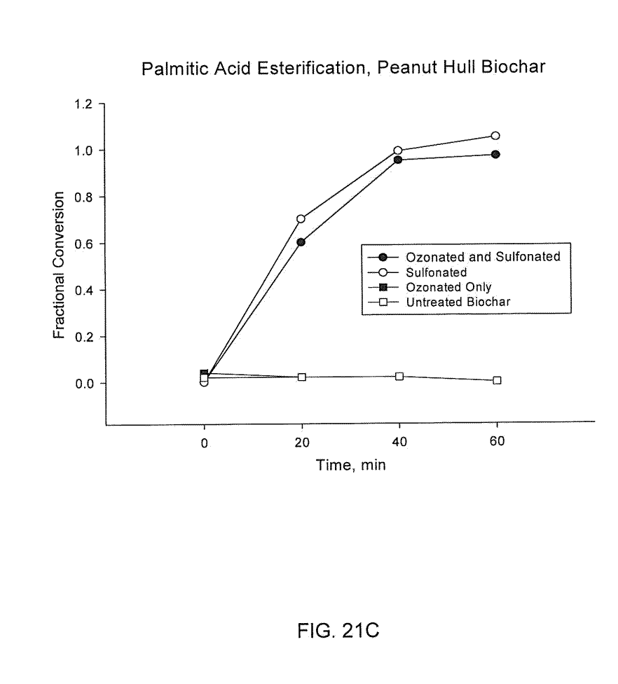

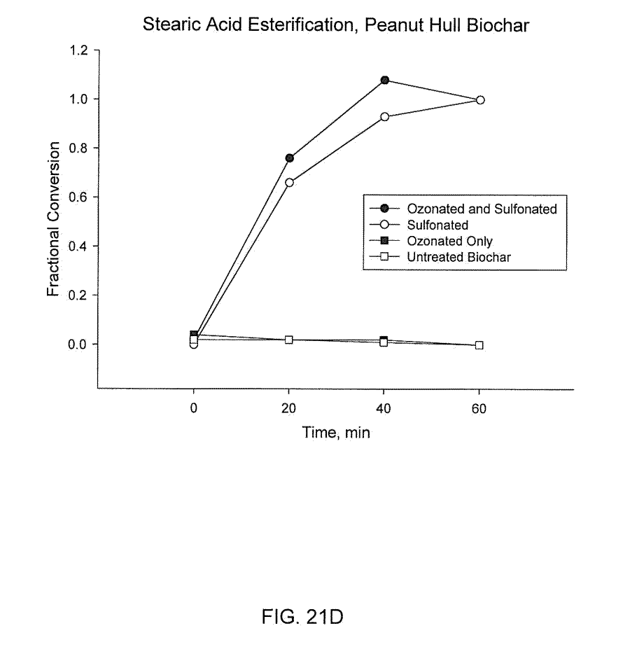

Control studies clearly indicated that sulfonation of the chars was required for catalytic activity and esterification did not take place without the catalysts. Controls consisted of a blank (methanol plus palmitic or stearic acid) and non-sulfonated chars (char plus palmitic or stearic acid and methanol). Reactions (0.10 g peanut hull char, 5 mL methanol, and 200 ppm of palmitic or stearic acid) conducted at 50° C. for 4 hours (data not shown) or 58° C. for 1 h (FIG. 21) indicated no formation of methyl esters, contrary to positive controls using HCl (1 drop 38% HNO₃) under identical conditions, which resulted in 100% conversion of the fatty acids to methyl esters (data not shown).

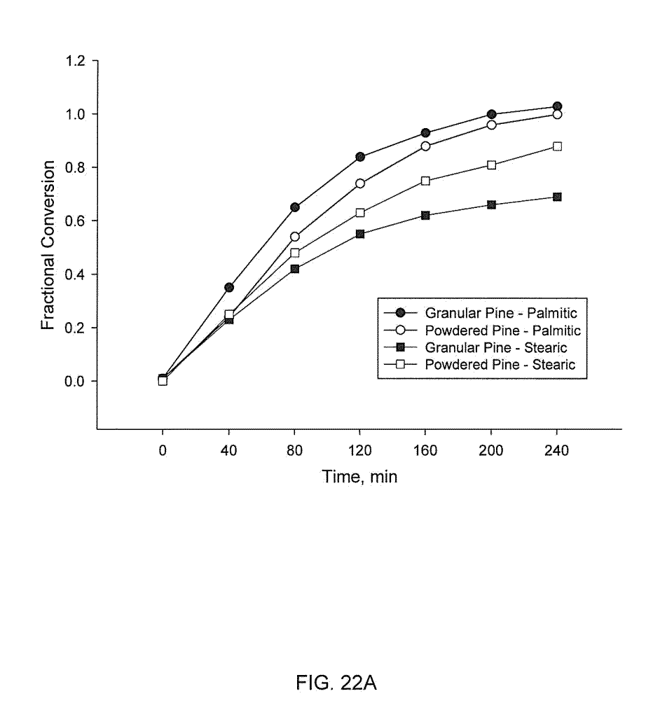

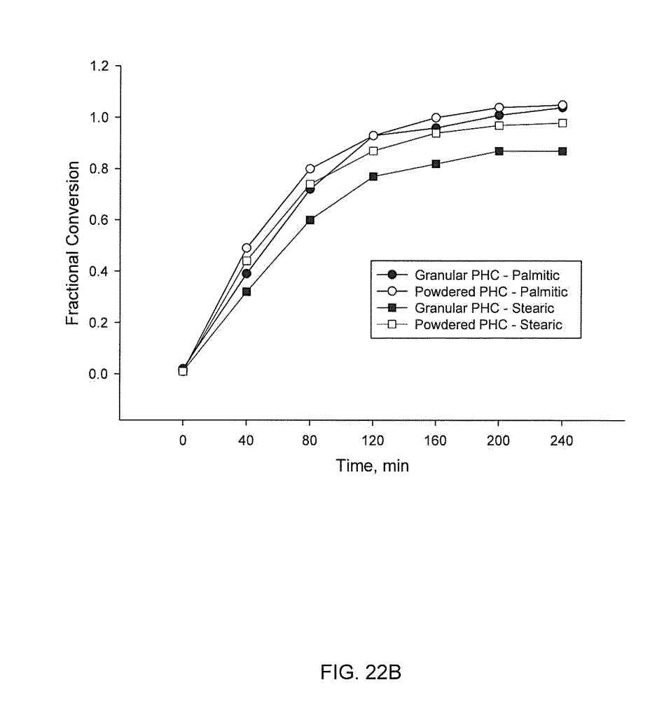

All sulfonated chars were demonstrated to be catalytically active for esterification of palmitic and stearic acid with methanol. Chars treated with ozone only (i.e., weak acid functional groups on the surface, e.g., carboxylic acid groups) were not active and chars ozonated followed by sulfonation resulted in esterification activities similar to chars that were directly sulfonated (FIG. 21). The esterification rate and fractional conversion of palmitic and stearic acid were similar, but in the case of stearic acid appeared to be reduced when using granular catalysts (FIG. 22). Solid acid catalysts generated from pelletized peanut hulls and pine pellets did not show significant differences in catalytic activity (FIG. 22).

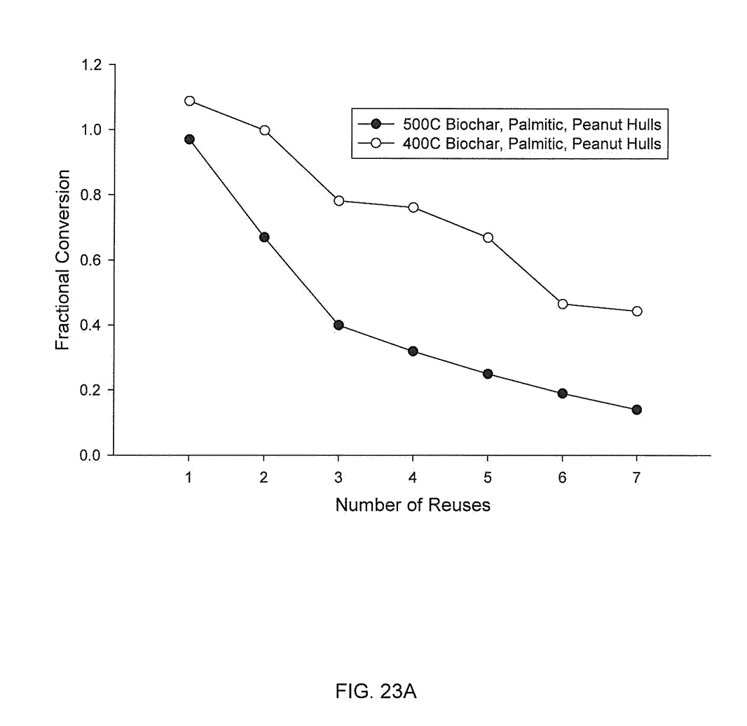

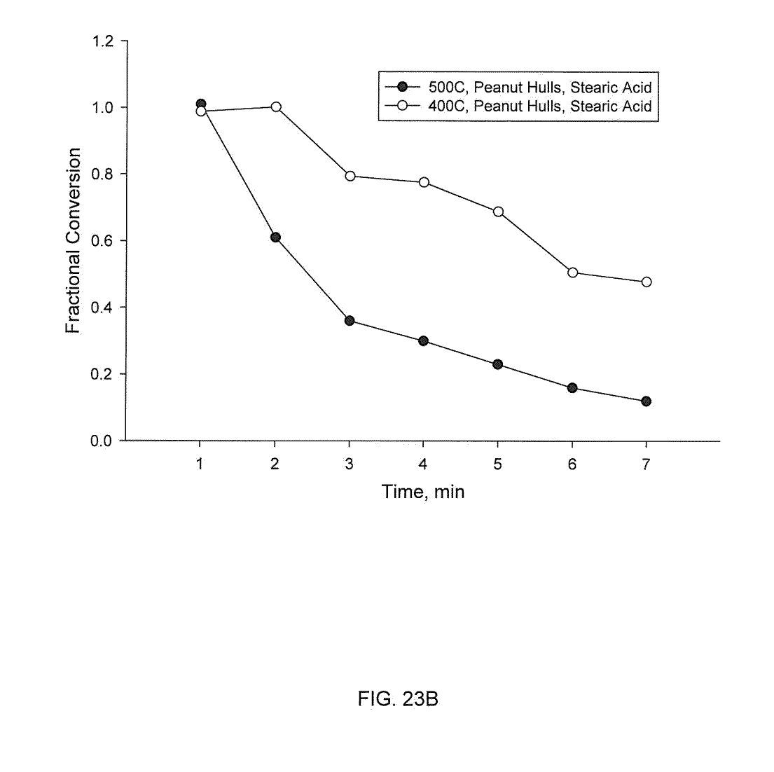

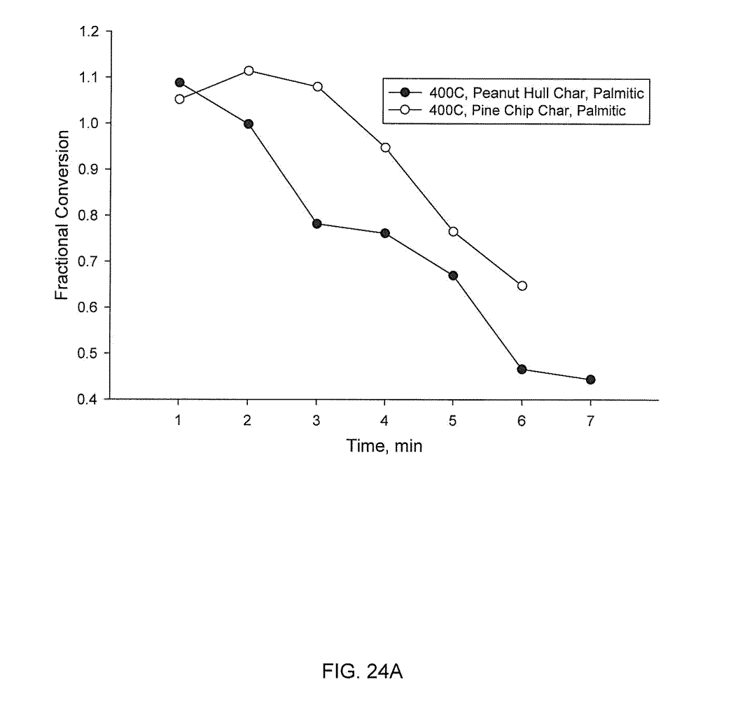

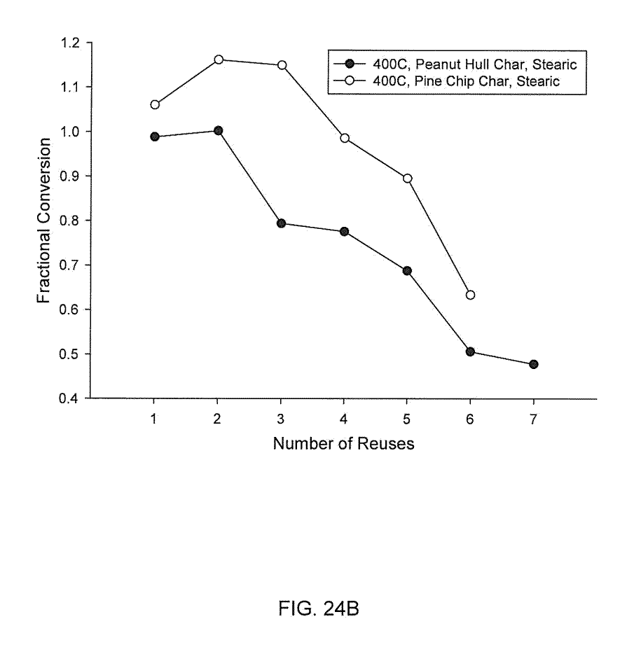

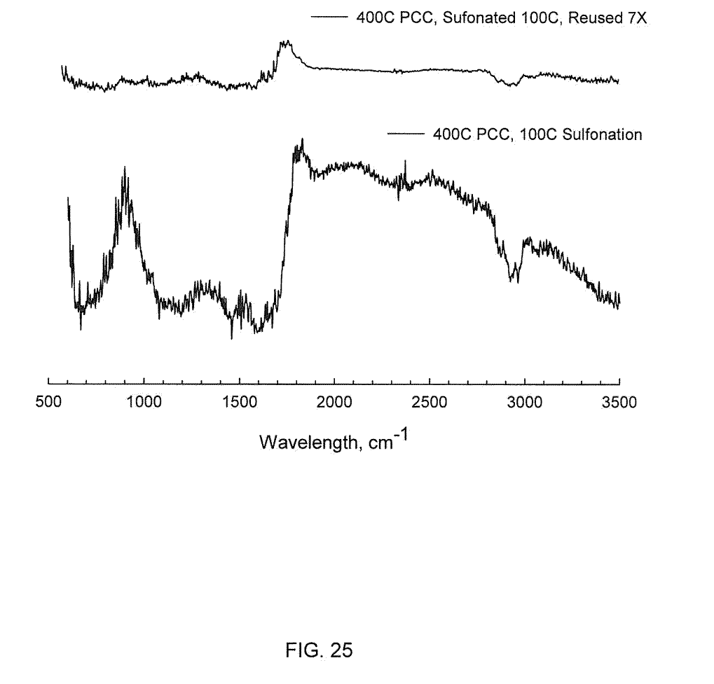

Catalytic activity appeared to follow the trends implied by the DRIFT analysis of the synthesized catalysts. Chars synthesized at lower temperatures and sulfonated at lower temperatures (400° C. for pyrolysis, 100° C. for sulfonation) had the highest activity (FIG. 23). Additionally, sulfonated char generated from pine chips (not pellets) appeared to maintain higher esterification activity upon reuse relative to peanut hull char (FIG. 24) and repeated reuse of the catalysts indicated a reduction in activity, but not a complete loss in activity (FIGS. 23 and 24). DRIFT analysis of the reused char did indicate a loss in the peaks representative of —SO₃ functional groups (FIG. 25), but it is not clear from these data whether this was due to leaching of the active sites or adsorption of poisons on the surface of the catalyst.

Acid Catalyst Regeneration and Reuse

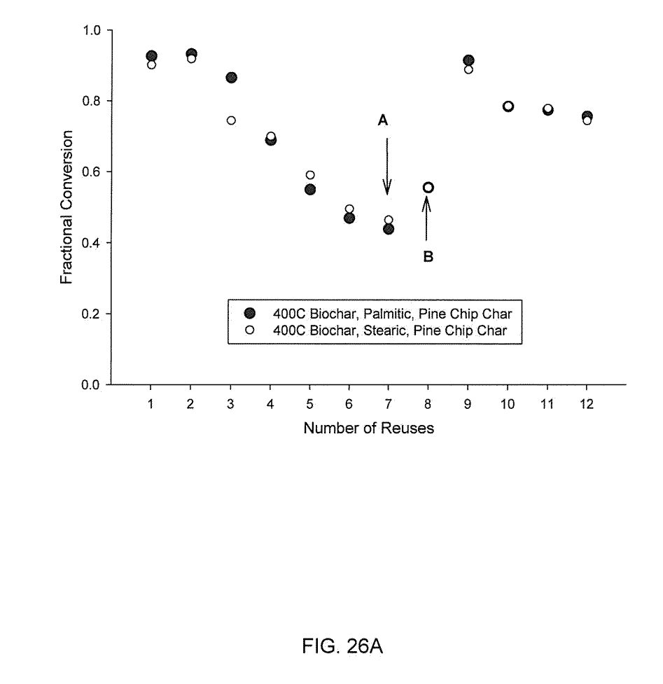

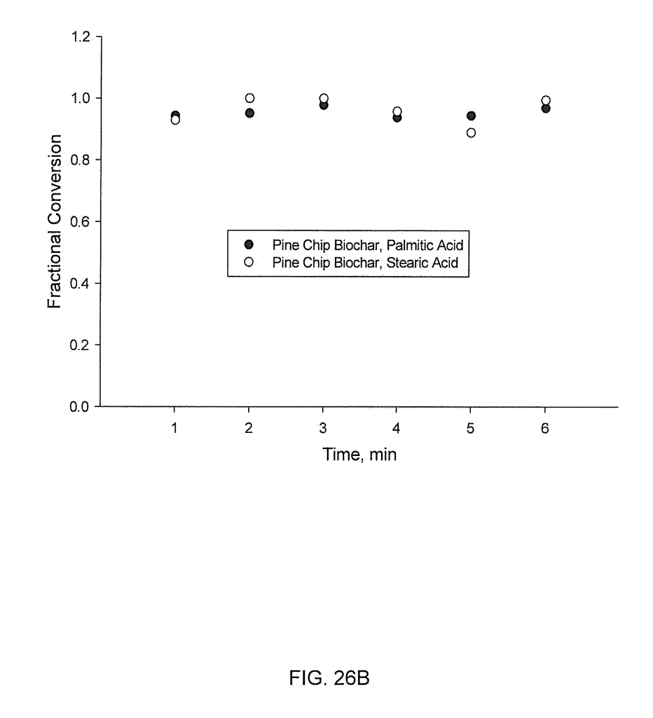

Since catalytic activity declined with repeated use of the catalyst, different regeneration techniques were explored to determine if activity could be regenerated. Clearly, rinsing the char catalyst with methanol in between batch reactions was not effective, since this resulted in declining activity (FIGS. 23 and 24). Subsequently, contacting the char catalyst with heptane and then drying at 125° C. or 250° C. was determined to significantly increase catalytic activity of the char catalyst (FIG. 26). Finally, drying the acid catalyst at 125° C. (1 h) between uses, without solvent contact, resulted in a consistent, high catalytic activity with repeated uses of the char catalyst (FIG. 26).

Solid Acid Esterification Activity in Fats and Oils

Catalytic testing of the solid acid catalyst generated from biochar indicated that sulfonated biochar (100° C., 12 h) generated from 400° C. pine chip biochar had the most robust esterification activity and thus, was tested for activity in fats and oils. Conversion of FFAs in oil or poultry fat (10-14%) ranged between 50 to 80% (65° C., 2 h) measured using GC/FID methods and approached 100% when measured using the total acid number (Table 8). Increasing the methanol to FFA ratio also increased the measured conversion of FFAs to methyl esters (Table 8).

Analysis of Solid Acid Catalysts Relative to Literature

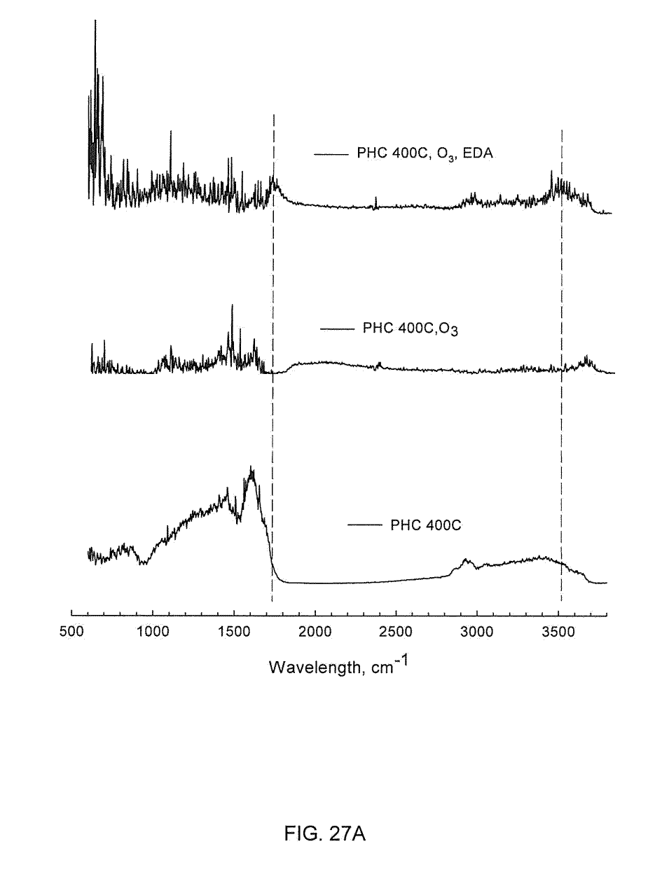

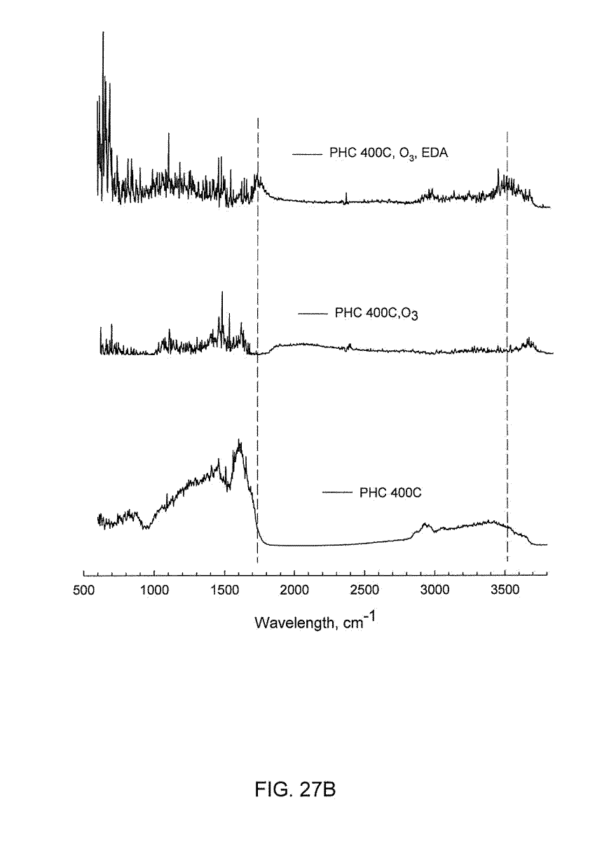

Recently, solid acid, carbon catalysts (e.g., attached —SO₃H groups) have been generated from refined sugars (e.g., pure cellulose, glucose and starch) and demonstrated to catalyze esterification reactions (Nature. 2005, 438, 178, which is herein incorporated by reference for the corresponding discussion). In order to generate the solid acid catalysts, the refined carbohydrates are pyrolyzed at low temperatures (400-500° C.) to generate a cross-linked, polyaromatic polymer that is subsequently sulfonated using concentrated H₂SO₄ (or other sulfonation agents). Our results indicate that solid acid catalysts can be generated from crude biomass, contrary to refined carbohydrates, using a slow, low temperature pyrolysis process (1-20° C./min, 400-500° C.). In our process, during low temperature pyrolysis of biomass, hemicellulose is thermally decomposed and partially volatilized, and cellulose and lignin undergoes a series of thermal/catalytic (potassium and calcium salts present in the biomass catalyze this process) reactions leading to a polyaromatic structure that can be sulfonated (Fuel. 2004, 83, 1469-1482, which is herein incorporated by reference for the corresponding discussion). DRIFT analysis of the biochars (FIG. 27) indicated bands at 1100-1500 cm⁻¹ (aromatic C═C vibrations), 1600 cm⁻¹ (1590 cm⁻¹, aromatic rings) and 3000 cm⁻¹ (aromatic C—H stretch), and when coupled with literature analysis provide evidence for polycyclic aromatic polymer formation (Id.).

Overall, our results are comparable to those reported in the literature for solid acid catalysts derived from refined carbohydrates. The surface area of the biochar (2-4 m²/g) is similar to the 1-8 m²/g reported for carbonized and sulfonated refined carbohydrates (Bioresource Technology. 2008, 99, 8752-8758; Catalysis Today. 2006, 116, 157-161, which are herein incorporated by reference for the corresponding discussion), but pore size was significantly smaller (0.004 vs. 0.4-0.8 cm³/g—Bioresource Technology. 2008, 99, 8752-8758, which is herein incorporated by reference for the corresponding discussion). It is possible that sulfonation of the biochar may have altered the surface area and pore size of the resultant solid acid catalyst, but we did not measure for this effect. Acid density based on elemental sulfur analysis (Table 7) was significantly higher in the pine chip char and potentially accounted for the higher esterification activity of this solid acid catalyst. The measured acid density in our catalysts were on average lower (0.3-1.12 mmol SO₃H/g), but comparable to solid acid catalysts derived from glucose (0.7-1.5, J. Am. Ceram. Soc. 2007, 90, 12, 3725-3734; Bioresource Technology. 2008, 99, 8752-8758; Catalysis Today. 2006, 116, 157-161, which are herein incorporated by reference for the corresponding discussion), starch (1.8, Bioresource Technology. 2008, 99, 8752-8758, which is herein incorporated by reference for the corresponding discussion), glucose impregnated carbonized polymer (0.7-2.4, Mo et al., 2008), and cellulose (1.7, Lou et al., 2008). The higher acid density in the sulfonated pine chip char and overall lower acid density compared with acid catalysts prepared from refined carbohydrates may have been due to the larger particle size of the starting biomass (8 mm dia., peanut hull pellets; 1-5 mm thick pine chips). The biomass particles we have used may not be conducive to uniform or complete pyrolysis (due to temperature gradients within the particles) and complete formation and cross linking of the polycyclic aromatic sheets from cellulose and lignin in the biomass may not have occurred. These aromatic sheets are the anticipated sites for sulfonation and active sites in the catalysts. Our particle size (e.g., about 8 mm) compares to Suganuma et al., 2008 who demonstrated hydrolysis of pure microcrystalline cellulose (Avicel) with a solid acid catalyst generated from low temperature, pure cellulose pyrolysis using very small Avicel particles (20-100 μm particles, 450° C. pyrolysis, 5 h) (J. Am. Chem. Soc., 2008, 130, 12787-127793, which is herein incorporated by reference for the corresponding discussion).

It is difficult to compare catalytic activities with the literature, since esterification reaction rates and conversions are a function of temperature, substrate concentration, and catalyst loading and active site density, and in most cases are different from study to study. One option is to compare calculated reaction rates assuming a constant temperature, volume, and catalyst mass, using a batch reactor design equation,

$$- r = \frac{{VC}_{Ao}}{W} \times \frac{Δ X}{Δ t} \qquad ( 3 )$$

where −r is the rate of esterification (μmol g⁻¹ min⁻¹), V the volume (L), C_{Ao} the initial concentration of fatty acid (μmol L⁻¹), or C_{Ao} V the initial mass of fatty acid (mg), ΔX the change in fractional conversion over Δt, and Δt the change in time (min). Using equation 3, initial esterification reaction rates were calculated from the batch data (data within the first 20-60 min) and compared to the literature. Esterification reaction rates for palmitic and stearic acids using solid acid pine and peanut hull catalysts ranged from 0.35 to 0.77 μmol g-cat⁻¹ min⁻¹ (using data from FIGS. 21 and 22, 55-58° C., 1-2.5 mg fatty acid). Lou et al., report oleic acid esterification rates ranging from 500-700 μmol g-cat⁻¹ min⁻¹ using catalysts derived from glucose, cellulose, and starch (80° C., 2820 mg oleic acid). Takagaki et al., 2006 report an ethyl oleate esterification rate between 220 to 430 μmol g-cat⁻¹ min⁻¹ using refined carbonized glucose (80° C., 3100 mg oleic acid). The lower reactions rates in our work were probably due to lower temperature (55-58 vs. 80° C.) and initial fatty acid concentration (2 vs. 3000 mg), since reaction rates are directly proportional to temperature and in many cases increase with increasing substrate concentrations. Kinetic analysis of fatty acid esterification using acid ion-exchange resins indicate that the rate law (algebraic equation that describes reaction rates) can be fit to a second order model and is proportional to the mole fraction (i.e., concentration) of the fatty acid and alcohol (Ind. Eng. Chem. Res. 2005, 44, 7978-7982, which is herein incorporated by reference for the corresponding discussion).

Catalytic activity among the different solid acid carbon catalysts can also be compared by analyzing percent or fractional conversion of the FFAs to methyl esters in a defined period. Thus, we also compared fractional conversions of FFAs in spiked oils or waste oils using solid acid catalysts (Table 8). Fractional conversions of palmitic and stearic acid in spiked soybean oil and poultry fat were similar (50-80%, 2 h, 65° C.) to conversions reported for catalysts generated from pyrolyzed starch and glucose (20-60%, 1-2 h, 60-80° C.), and commercial resins. Recently, two commercial resins (Amberlyst BD20 and Dowex 550A, a strong base anion exchange resin) have been tested for their ability to esterify FFAs in oil (Table 8). Conversions ranged from 62-90% in a two hour period from 55-80° C. for the commercial resins (Table 8). One acknowledged drawback to our work is the high methanol to FFA ratios at which the catalytic reactions were performed. Increasing methanol to FFA ratios increase yields or conversion to methyl esters and it is distinctly possible that fractional conversions using our catalysts would be lowered when conducting the reactions at more industrially relevant methanol to FFA ratios (at the same conditions −3-6% catalyst, 65° C., 2 h, 10-15% FFAs).

Key to industrial use of solid acid catalysts is their ease of recovery and reusability. As previously noted, repeated reuse of our solid acid catalysts, without regeneration between steps, resulted in a decline in activity, with deactivation more severe in the peanut hull biochar catalyst. The continued decline in fractional conversion and loss of an IR signal representative of the —SO₃H group (FIGS. 23, 24, 25), upon recovery and reuse of the solid acid catalysts, originally suggested that leaching of active sites was responsible for the decline in activity. Catalytic activity continued to decline or was not fully recovered even after washing with solvents such as methanol and heptane, indicating bound fatty acids were not inhibiting the reaction. However, when the catalysts were dried between use (125° C.), activity remained >90% (FIG. 26). These results suggest that water adsorption may have caused the decline in activity in our catalysts and not leaching of sulfonated PAH fragments from the catalytic biochar. Similar to our results, Mo et al., 2008, synthesized a solid acid carbon catalyst via glucose impregnation of Amberlite XAD1180, followed by low temperature pyrolysis (300° C.) and sulfonation (concentrated H₂SO₄ was added prior to pyrolysis). This catalyst when recovered and dried at 100° C. between uses maintained catalytic esterification activity for 6 cycles, unlike sulfonated amorphous carbon generated from glucose (Catal Lett. 2008, 123, 1-6, which is herein incorporated by reference for the corresponding discussion). Similarly, solid acid catalysts generated from pyrolyzed expanded starch (Starbon®) or ordered mesoporous carbon (OMC), when washed in acetone or water and dried between reuse maintained activity for at least 3-5 cycles (Chem. Commun. 2007, 634-636, which is herein incorporated by reference for the corresponding discussion).

However, three research groups recently reported maintaining esterification activity of solid acid catalysts without regeneration (the catalysts were simply filtered from the reaction mixture and reused). Lou et. al., 2008 and Takagaki et al., 2006 report that solid acid carbon catalysts generated from starch and glucose, respectively, can be reused multiple times without a regeneration step in the esterification of oleic acid with ethanol and waste cooking oils with methanol. Park et al., 2010 report similar results using a newly synthesized Amberlyst BD20 solid acid catalyst. All of these researchers performed their catalytic reactions at 80° C. (significantly higher than our work and others at 55-65° C.—Table 8), which may account for the different regeneration requirements. Reaction temperatures at 80° C. may have prevented (or minimized) significant adsorption of water on the catalyst surface, thereby preventing reaction inhibition. Interestingly, the Amberlyst BD20 catalyst was treated with distilled water, 50, 70, and 95 wt % ethanol, ethanol, acetone, and petroleum ether in series before catalytic testing. It is not clear why this solvent pretreatment was performed, whether it is needed to activate the catalyst, nor has the chemical structure of the catalyst been reported (Bioresource Technology. 2010, 101, S62-S65, which is herein incorporated by reference for the corresponding discussion).

Conclusions

Biochars generated by biomass pyrolysis at 400° C. and sulfonated at 100° C. were successful, solid acid carbon supported catalysts, highly active for esterification of palmitic and stearic acid with methanol, indicative of sulfonic acid groups (—SO₃) acting as the catalytic site. Drying the solid acid carbon supported catalyst (125° C., 1 h) maintained esterification catalytic activity indicating bound water may have inhibited esterification activity. These results indicate that solid acid catalysts can be generated from biomass and renewable carbon sources via low temperature pyrolysis without the need to refine the biomass.

TABLE 7
Compositional analysis of solid acid catalysts.

| | | | | SO₃H |
|---|---|---|---|---|
| | | | | Density |
| Catalysts | Carbon, % | Nitrogen, % | Sulfur, % | mmol/g^{a} |
| PHC-500P | 64 ± 1.7 | 2.0 ± 0.30 | 0.14 ± 0.05 | |
| PCC-400P | 65 ± 0.38 | 1.8 ± 0.6 | 0.10 ± 0.03 | |
| PHC-400P- | 68 ± 1.1 | 2.0 ± 0.10 | 2.1 ± 0.04 | 0.61 ± 0.03 |
| 150S | | | | |
| PCC-400P- | 56 ± 0.30 | 0.24 ± 0.01 | 3.7 ± 0.08 | 1.12 ± 0.02 |
| 100S | | | | |
| PPC-400P- | 62 ± 0.76 | 0.40 ± 0.08 | 1.2 ± 0.11 | 0.34 ± 0.01 |
| 100S | | | | |
| PCC-400P- | 58 ± 0.23 | 0.39 ± 0.13 | 2.30 ± 0.08 | 0.69 ± 0.006 |
| 100S | | | | |
| PCC-400P- | 46 ± 17.4 | 0.31 ± 0.03 | 1.39 ± 0.35 | 0.41 ± 0.08 |
| 150S | | | | |
| PCC-400P- | 62 ± 20.5 | 0.43 ± 0.23 | 0.86 ± 0.30 | 0.24 ± 0.06 |
| 250S | | | | |

PHC, Peanut Hull Char;
PCC, Pine Chip Char;
PPC, pine pellet char
P, Pyrolysis Temperature;
S, Sulfonation Temperature
^{a}calculated from sulfur content assuming all S atoms are in the —SO₃H form with baseline sulfur content subtracted

TABLE 8
Free fatty acid conversions (palmitic and stearic acids) to methyl esters in
rendered poultry fat and soybean oil using a solid acid carbon catalyst (400° C. pine chip
biochar, sulfonated 100° C.) and comparison with literature. Catalytic tests were
performed at 65° C. for 2 hours using 2.0 grams of biochar catalyst.

| | | | | | | Residence | |
|---|---|---|---|---|---|---|---|
| | | | % | % | | Time, | Conversion |
| | | MeOH:FFA | FFA's | Catalyst | % | Temp. | Measurement |
| Catalyst | Fat or Oil | Molar Ratio | (w/w) | (w/w) | Conversion | (h, ° C.) | Method |
| PCC-400P- | Poultry | 39:1 | 13.9 | 5.6 | 49 ± 7 | 2, 65 | GC/FID |
| 100S | Fat | | | | | | |
| | | 55:1 | 10.5 | 3 | 98 ± 0.03 | 2, 65 | TAN* |
| | | | | | 81 ± 16 | 2, 65 | GC/FID |
| | | | | | 101.6 ± 0.02 | 2, 65 | TAN* |
| | Soybean | 39:1 | 13.5 | 5.6 | 53 ± 6.5 | 2, 65 | GC/FID |
| | Oil | | | | | | |
| Pyrolyzed- | Waste Oil | 20:1 | 27.8 | 10 | 60 | 2, 80 | GC/FID |
| Starch^{a} | | | | | | | |
| Amberlyst | Trap | 6:1 | 50 | 20 | 62 | 2, 80 | TAN |
| BD20^{b} | Grease | | | | | | |
| Pyrolyzed- | Palmitic- | 6:1 | 10 | 2 | 20 | 1, 60 | ¹H-NMR, Acid |
| Glucose^{c} | Soybean | | | | | | Titration |
| | Oil | | | | | | |
| Dowex | Oleic- | 6:1 | 10.7 | 2.3 | 80-90 | 2, 55 | Acid Titration |
| 550A^{d} | Sunflower | | | | | | |

*Conversion to methyl esters based on measurement of free fatty acids using potentiometric titration (ASTM 664), TAN is total acid number
^{a}Lou et al., 2008 - % conversion for esterification of FFA's and transesterification of triglycerides
^{b}Park et al., 2010
^{c}Mo et al., 2008 - solid acid, carbon catalyst via glucose impregnation of Amberlite XAD1180, followed by pyrolysis and sulfonation
^{d}Marchetti et al., 2007 - ethanol used in esterification

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of the disclosure. Many variations and modifications may be made to the above-described embodiments. All such modifications and variations are intended to be included herein within the scope of this disclosure and protected by the following claims.

Therefore, at least the following is claimed:

1. A solid acid catalyst, comprising:
a support; and
a plurality of acidic functional groups attached to the support.

2. The solid acid catalyst of claim 1, wherein the support is selected from the group consisting of: biochar, carbon, activated carbon, and a combination thereof.

3. The solid acid catalyst of claim 2, wherein the support is derived from biomass.

4. The solid acid catalyst of claim 3, wherein the biomass is selected from the group consisting of: peanut hull, wood chip, pine pellet, and a combination thereof.

5. The solid acid catalyst of claim 1, wherein the acidic functional groups are selected from the group consisting of: —SO₃H, —PO₃, and a combination thereof.

6. The solid acid catalyst of claim 1, wherein the catalyst is reusable.

7. A method of making a solid acid catalyst, comprising:
pyrolyzing biomass to form biochar; and
activating the biochar.

8. The method of claim 7, wherein the biomass is selected from the group consisting of: peanut hull, wood chip, pine pellet, and a combination thereof.

9. The method of claim 7, wherein the biomass is pyrolyzed at about 400° C. to 600° C.

10. The method of claim 7, wherein the biochar is activated with H₂SO₄.

11. The method of claim 10, wherein the biochar is activated at about 100° C. to 250° C.

12. The method of claim 7, further comprising:
drying the solid acid catalyst at about 100° C. to 125° C. for about 1 hour between uses to allow the solid acid catalyst to be reused.

13. A method of using a solid acid catalyst, comprising:
esterifying free fatty acids that are in contact with the solid acid catalyst for about 1 to 6 hours at about 25° C. to 60° C.
